SEQUENCE LISTING (4 sequences)

SEQ ID NO: 1 - DNA, 598 bp, Mus musculus
  misc_feature (1)..(598): Murine FIZZ1 nucleotide sequence
ggtacctaggtcagcaatcccatggcgtataaaagcatctcatctggccaggtcctggaacctttcctgagattctgccccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccagctttgatatgatgaatctaacaaaaactgcagtctcaatttggaaatctgactcatgtgcctttaaatgtgttcatattgcccatttaccctgcttcttgaaatgcttcttgaaaaataaagacaaatttgcatgtg

SEQ ID NO: 2 - protein, 101 aa, Mus musculus
  misc_feature (1)..(101): Murine FIZZ1 polypeptide
MKTTTCSLLICISLLQLMVPVNTDETIEIIVENKVKELLANIPANYPSTVTKTLSCTSVKTMNRWASCPAGMTATGCACGFACGSWEIQSGDTCNCLCLLV

SEQ ID NO: 3 - DNA, 594 bp, Homo sapiens
  misc_feature (1)..(594): Human FIZZ1 nucleotide sequence
ccacgttgtcttctttccttcaccaccacccaggagctcagagatctaagctgctttccatcttttctcccagccccaggacactgactctgtacaggatggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctgacagggaggaggctgagaactcagttttgtgaccatgacagtaatgaaaccagggtcccaaccaagaaatctaactcaaacgtcccacttcatttgttccattcctgattcttgggtaataaagacaaactttgtacctcaaaaaaaaaaaaaaaaaaaaa

SEQ ID NO: 4 - protein, 112 aa, Homo sapiens
  misc_feature (1)..(112): Human FIZZ1 polypeptide
MGPSSCLLLILIPLLQLINPGSTQCSLDSVMDKKKIKDVLNSLEYSPSPISKKLSCASVKSQGRPSSCPAGMAVTGCACGYGCGSWDVQLETTCHCQCSVVDWTTARCCHLT

(19) United States
(12) Patent Application Publication
(10) Pub. No.: US 2009/0274696 A1
(43) Pub. Date: Nov. 5, 2009

(54) METHODS FOR TREATING INFLAMMATION

(75) Inventors: Hang Chen, Framingham, MA (US); Michael R. Bowman, Westwood, MA (US); Bruce A. Jacobson, Framingham, MA (US); Lawrence Mason, Arlington, MA (US)

Correspondence Address: CHOATE, HALL & STEWART LLP/WYETH, PATENT GROUP, TWO INTERNATIONAL PLACE, BOSTON, MA, 02110, US

(73) Assignee: WYETH, Madison, NJ (US)

(21) Appl. No.: 12/432,617

(22) Filed: Apr. 29, 2009

Related U.S. Application Data
(60) Provisional application No. 61/126,131, filed on Apr. 29, 2008.

(51) Int. Cl. A61K 39/395; A61K 31/7052; A61P 29/00; C12Q 1/02; A61K 38/16; A61K 39/00; C07K 16/00

(52) U.S. Cl. 424/135.1; 514/44 A; 424/158.1; 424/136.1; 424/145.1; 424/133.1; 514/44 R; 435/29; 514/12; 424/184.1; 530/389.2

(57) ABSTRACT

Provided are methods and compositions for reducing airway hyperresponsiveness and other inflammatory diseases, disorders and conditions in a mammal by decreasing FIZZ1 (Found in Inflammatory Zone 1) activity. Also provided are methods and compositions for identifying modulators of airway inflammation and/or inhibitors of FIZZ1. The present invention encompasses modulators of airway inflammation and/or inhibitors of FIZZ1 and uses thereof. In addition, the present invention provides methods and compositions for enhancing an immune response based on FIZZ1 protein.

### RELATED APPLICATION INFORMATION

This application claims priority to and benefit of U.S. provisional application 61/126,131, filed on Apr. 29, 2008, the entire contents of which are herein incorporated by reference.

### BACKGROUND OF THE INVENTION

Inflammation is the complex biological response of vascular tissues to harmful stimuli, such as pathogens, damaged cells, or irritants. For example, asthma is associated with chronic inflammation of the airways. A hallmark feature of asthma is hyperresponsiveness of the airway smooth muscle to physical, chemical and environmental stimuli. This heightened responsiveness is associated with airway obstruction, as well as an increase in asthma severity and the need for drug therapy. Experimentation in the field of tracheal smooth muscle (TSM)-mediated hyperresponsiveness has largely focused on analysis of the cellular and molecular events induced by allergen exposure. In experimental animal models of airway hyperresponsiveness (AHR), as in human asthma, a variety of factors have been implicated in promoting inflammation and bronchoconstriction. Most of these factors are released from airway inflammatory cells and respiratory epithelial cells, which, in turn, act on the airway not only to amplify the inflammatory response but also to alter distinct signaling pathways resulting in the changes in the TSM functional properties observed in AHR.

### SUMMARY OF THE INVENTION

The present invention provides new methods for treating inflammation by targeting Found in Inflammatory Zone (FIZZ1) activity. The present invention is based on the discovery that FIZZ1, resistin-like molecule-α (a member of the resistin family of adipokines) is a new inflammatory mediator.

In one aspect, the present invention provides a method to reduce airway hyperresponsiveness in a mammal including a step of decreasing activity of Found in Inflammatory Zone (FIZZ1).

In some embodiments, the step of decreasing the activity of FIZZ1 includes reducing FIZZ1 activity in tracheal smooth muscle of the mammal. In some embodiments, the step of decreasing the activity of FIZZ1 includes reducing FIZZ1 activity in airway epithelium.

In some embodiments, the airway hyperresponsiveness treated by the method of this aspect of the invention is associated with asthma.

In another aspect, the present invention provides a method for treating inflammation including a step of decreasing FIZZ1 activity in a mammal in need of treatment. In some embodiments, the inflammation treated by methods of the invention is in a digestive, pulmonary or reproductive tract. In some embodiments, the step of decreasing the FIZZ1 activity includes reducing the FIZZ1 activity in an epithelial barrier of the digestive, pulmonary or reproductive tract.

In some embodiments, the inflammation treated by methods of the invention is airway inflammation. In some embodiments, the step of decreasing the FIZZ1 activity includes reducing the FIZZ1 activity in airway epithelium. In some embodiments, the step of decreasing the FIZZ1 activity includes reducing the FIZZ1 activity in tracheal smooth muscle of the mammal. In some embodiments, the airway inflammation treated is associated with asthma.

In some embodiments, the inflammation treated by methods of the invention is induced by allergen.

In some embodiments, the inflammation treated by the methods of the invention is associated with cardiovascular diseases or disorders; neurodegenerative diseases such as, Alzheimer's; infectious diseases, such as, for example, myocarditis, cardiomyopathy, acute endocarditis, pericarditis; atherosclerosis; Systemic Inflammatory Response Syndrome (SIRS)/sepsis; adult respiratory distress syndrome (ARDS); asthma; rheumatoid arthritis; osteoarthritis; systemic erythematosis (SLE); Airway hyperresponsiveness (AHR); bronchial hyperreactivity; Chronic Obstructive Pulmonary disease (COPD); Crohn's disease; Congestive Heart Failure (CHF); inflammatory bowel disease; inflammatory complications of diabetes mellitus; metabolic syndrome; end-stage renal disease (ESRD); muscle fatigue or inflammation and dermal conditions; or inflammatory conditions caused by bacterial infection or viral infection.

In some embodiments, the step of decreasing the FIZZ1 activity includes reducing transcription of FIZZ1 gene. In some embodiments, the step of decreasing the FIZZ1 activity includes reducing translation of an mRNA sequence encoding FIZZ1 protein.

In some embodiments, the activity of FIZZ1 is decreased by administering to the mammal an interfering RNA. In some embodiments, the interfering RNA is selected from siRNA, shRNA or miRNA. In some embodiments, the interfering RNA is siRNA. In some embodiments, the siRNA suitable for the invention includes a sequence substantially complementary to at least a portion of the mRNA encoding the FIZZ1 protein. In some embodiments, the siRNA is double-stranded. In some embodiments, the siRNA is single-stranded. In some embodiments, the siRNA suitable for the invention includes a sequence having between about 20 and about 25 nucleotide bases.

In some embodiments, the step of decreasing the FIZZ1 activity includes administering to the mammal an antibody, or a fragment thereof, that specifically binds the FIZZ1 protein. In some embodiments, the antibody, or a fragment thereof, is selected from the group consisting of intact IgG, F(ab′)2, F(ab)₂, Fab′, Fab, ScFv, single domain antibodies, diabodies, triabodies and tetrabodies. In some embodiments, the antibody suitable for the invention is a monoclonal antibody. In some embodiments, the antibody suitable for the invention is a humanized monoclonal antibody. In some embodiments the antibody is a single chain antibody. In some embodiments, the step of decreasing FIZZ1 activity comprises administering an FIZZ1 binding protein. In some embodiments, the FIZZ1 binding protein suitable for the invention is a single domain binding protein. In some embodiments, the FIZZ1 binding protein suitable for the invention is an IgNAR, a VHH or a SMIP™.

In some embodiments, the step of decreasing the FIZZ1 activity includes administering to the mammal an aptamer that specifically binds the FIZZ1 protein. In some embodiments, the aptamer is an RNA aptamer.

In some embodiments, the step of decreasing the activity of FIZZ1 includes administering to the mammal a small molecule that inhibits FIZZ1 activity.

In yet another aspect, the present invention provides a method for evaluating the ability of an agent to modulate airway inflammation. The method includes the steps of: (1) providing a trachea sample; (2) culturing the trachea sample in a medium in the presence of FIZZ1; (3) providing an agent to the medium; (4) determining the histology of the trachea sample; and (5) comparing the histology result from step (4) to a control to evaluate the ability of the agent to modulate airway inflammation.

In some embodiments, step (4) includes determining the histological intactness of the epithelial layer in the trachea sample. In some embodiments, the control includes the histology of a tracheal sample cultured in the medium in the absence of FIZZ1. In some embodiments, the control includes the histology of a tracheal sample cultured in the medium in the presence of FIZZ1. In some embodiments, the trachea sample is derived from a mouse. In some embodiments, the method further includes a step of identifying a modulator of airway inflammation based on the comparison result from step (5).

In still another aspect, the present invention provides a method for evaluating the ability of an agent to modulate airway hyperresponsiveness. The method includes the steps of: (1) providing a trachea sample; (2) culturing the trachea sample in a medium in the presence of FIZZ1; (3) providing an agent to the medium; (4) providing carbachol to the medium; (5) determining a contractile response to carbachol of the trachea sample; and (6) comparing the contractile response to carbachol determined in step (5) to a control to evaluate the ability of the agent to modulate airway hyperresponsiveness.

In some embodiments, the control includes the contractile response to carbachol of a tracheal sample cultured in the medium in the absence of FIZZ1. In some embodiments, the control includes the contractile response to carbachol of a tracheal sample cultured in the medium in the presence of FIZZ1. In some embodiments, the trachea sample is derived from a mouse. In some embodiments, the method further includes a step of identifying a modulator of airway hyperresponsiveness based on the comparison result from step (6).

In a further aspect, the present invention provides a method of screening inhibitors of FIZZ1. The method includes the steps of: (1) providing a plurality of trachea samples, each of which is cultured in a medium in the presence of FIZZ1; (2) providing a plurality of inhibitor candidates; (3) determining a phenotype associated with FIZZ1-mediated airway inflammation or hyperresponsiveness in each of the plurality of trachea samples; (4) comparing the phenotype determined in step (3) to a control; and (5) identifying one or more inhibitors of FIZZ1 that reduce the phenotype based on the comparison result in step (4).

In some embodiments, the plurality of inhibitor candidates include a small molecule library. In some embodiments, the plurality of inhibitor candidates include an antibody library. In some embodiments, the antibody library suitable for a method of this aspect of the invention is a single chain Fv library. In some embodiments, the plurality of inhibitor candidates include an peptide or protein library containing candidate FIZZ1-binding proteins (e.g., single domain binding proteins, IgNAR, VHH or SMIP™ proteins). In some embodiments, the plurality of inhibitor candidates include an interfering RNA library. In some embodiments, the plurality of inhibitor candidates include an aptamer library (e.g., an RNA aptamer library). In some embodiments, step (3) includes determining the histology of each of the plurality of trachea samples. In some embodiments, step (3) includes determining contractile response to carbachol.

The present invention further provides inhibitors of FIZZ1 identified according to the methods described in various embodiments above. In some embodiments, the present invention provides small molecule inhibitors of FIZZ1 identified according to the methods described in various embodiments above.

In still another aspect, the present invention provides a method for enhancing an immune response in a mammal. The method includes administering a polypeptide encoding FIZZ1 protein (SEQ ID NO:4), a fragment thereof, or a variant having at least 90% sequence identity to the FIZZ1 protein (SEQ ID NO:4).

In yet another aspect, the present invention provides a vaccine containing a polypeptide encoding FIZZ1 protein (SEQ ID NO:4), a fragment thereof, or a variant having at least 90% sequence identity to the FIZZ1 protein (SEQ ID NO:4).

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### DEFINITIONS

Agent: As used herein, the term “agent” refers to any compound or composition that can be tested as a potential modulator. Examples of agents that can be used include, but are not limited to, a small molecule, an antibody, antibody fragment, siRNA, shRNA, nucleic acid molecule (RNA or DNA), antisense oligonucleotide, a ribozyme, peptide, peptide mimetic, and the like. In some embodiments, an agent can be isolated or not isolated. As a non-limiting example, an agent can be a library of agents. If a mixture of agents is found to be a modulator, the pool can then be further purified into separate components to determine which components are in fact modulators of a target activity.

Airway hyperresponsiveness: As used herein, the term “airway hyperresponsiveness” (AHR) refers to an abnormality of the airways that allows them to narrow too easily and/or too much in response to a stimulus capable of inducing airflow limitation. AHR can be a functional alteration of the respiratory system caused by inflammation or airway remodeling (e.g., such as by collagen deposition). Airflow limitation refers to narrowing of airways that can be irreversible or reversible. Airflow limitation or airway hyperresponsiveness can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma, abnormalities in neural regulation of smooth muscle function (including adrenergic, cholinergic and nonadrenergic-noncholinergic regulation), and infiltrative diseases in and around the airways. AHR can be measured by a stress test that comprises measuring a mammal's respiratory system function in response to a provoking agent (i.e., stimulus). AHR can be measured as a change in respiratory function from baseline plotted against the dose of a provoking agent. Respiratory function can be measured by, for example, spirometry, plethysmograph, peak flows, symptom scores, physical signs (i.e., respiratory rate), wheezing, exercise tolerance, use of rescue medication (i.e., bronchodialators) and blood gases. In particular, AHR can be measured as lung resistance (RL) in vivo or the ex vivo force response of TSM tissue.

Allergen: As used herein, the term “allergen” refers to a substance (including antigen) that can induce an allergic or asthmatic response in a susceptible subject. The list of allergens can include proteins (e.g., ovalbumin), pollens, insect venoms, animal dander dust, fungal spores and drugs (e.g. penicillin). Examples of allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Amelioration: As used herein, the term “amelioration” is meant the prevention, reduction or palliation of a state, or improvement of the state of a subject. Amelioration includes, but does not require complete recovery or complete prevention of a disease condition. For example, amelioration may be considered to be at least about 30%, at least about 50%, at least about 70%, at least about 80%, and at least about 90% reduction in the levels of inflammatory markers associated with inflammation or an inflammatory condition or a reduction in the symptoms associated with inflammation such as for example, pain and/or edema associated with inflammation.

Antibodies: As used herein, the term “antibodies” is intended to include immunoglobulins and fragments thereof which are specifically reactive to the designated protein or peptide, or fragments thereof. Suitable antibodies include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), and antibody fragments. As used herein, the term “antibodies” also includes intact monoclonal antibodies, polyclonal antibodies, multi-specific antibodies (e.g. bi-specific antibodies) formed from at least two intact antibodies, and antibody fragments so long as they exhibit the desired biological activity.

Antibody fragment: As used herein, an “antibody fragment” includes a portion of an intact antibody, such as, for example, the antigen-binding or variable region of an antibody. Examples of antibody fragments include the Fab, Fab′, F(ab′)2, and Fv fragments of an intact antibody.

Binding protein: As used herein, the term “binding protein” includes any naturally occurring, synthetic or genetically engineered protein that binds an antigen or a target protein or peptide. Binding proteins can be derived from naturally occurring antibodies or synthetically engineered. A binding protein can function similarly to an antibody by binding to a specific antigen to form a complex and elicit a biological response (e.g., agonize or antagonize a particular biological activity). Binding proteins can include isolated fragments, “Fv” fragments consisting of the variable regions of the heavy and light chains of an antibody, recombinant single chain polypeptide molecules in which light and heavy chain variable regions are connected by a peptide linker (“ScFv proteins”), and minimal recognition units consisting of the amino acid residues that mimic the hypervariable region.

Carbachol: As used herein, the term “carbachol” (also known as carbamylcholine) includes carbachol (a choline ester) and its derivatives that capable of binding and stimulating acetylcholine receptors (e.g., muscarinic and nicotinic receptors).

Complementary: As used herein, the terms “complementary” or “complement(s)” refer to nucleic acid(s) that are capable of base-pairing according to the standard Watson-Crick, Hoogsteen or reverse Hoogsteen binding complementarity rules.

Diabodies: As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

Hybridization: As used herein, the terms “hybridization,” “hybridizes” or “capable of hybridizing” refer to the forming of a double or triple stranded molecule or a molecule with partial double or triple stranded nature.

Inflammation: As used herein, the terms “inflammation” or “inflammatory conditions” refer to the biological response of vascular tissues (e.g., digestive, pulmonary or reproductive tracts) to harmful stimuli, such as pathogens, damaged cells, or irritants, including one or more biological and physiological sequelae such as vasodilatation; increased vascular permeability; extravasation of plasma leading to interstitial edema; chemotaxis of dendritic cells, eosinophils, basophils, neutrophils, macrophages and lymphocytes; cytokine production; acute phase reactants; C-reactive protein (CRP); increased erythrocyte sedimentation rate; leukocytosis; fever; increased metabolic rate; impaired albumin production and hypoalbuminemia; activation of complement; activation of mast cells; stimulation of antibodies and the like.

Inflammation diseases, disorders or conditions: As used herein, the term “inflammation diseases, disorders or conditions” includes, by way of non-limiting example, arthritis (including rheumatoid arthritis, juvenile rheumatoid arthritis, osteoarthritis, psoriatic arthritis, lupus-associated arthritis or ankylosing spondylitis); scleroderma; systemic lupus erythematosis; HIV; Sjogren's syndrome; vasculitis; multiple sclerosis; autoimmune thyroiditis; asthma (e.g., allergic and non-allergic asthma); dermatitis (including atopic dermatitis and eczematous dermatitis); myasthenia gravis; inflammatory bowel disease (IBD); Crohn's disease; colitis; diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); sarcoidosis; scleroderma; cirrhosis; eosinophilic esophagitis; cardiovascular disorders (e.g., cholesterol metabolic disorders, oxygen free radical injury, ischemia, pulmonary fibrosis, idiopathic pulmonary fibrosis); disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease (e.g., myocarditis, cardiomyopathy, acute endocarditis, pericarditis); Systemic Inflammatory Response Syndrome (SIRS)/sepsis; atopic disorders, e.g., urticaria, allergic rhinitis, rhinosinusitis (e.g., chronic allergic rhinosinusitis) allergic enterogastritis; adult respiratory distress syndrome (ARDS); systemic erythematosis (SLE); Airway hyperresponsiveness (AHR); bronchial hyperreactivity; Chronic Obstructive Pulmonary disease (COPD); Congestive Heart Failure (CHF); inflammatory bowel disease; inflammatory complications of diabetes mellitus; metabolic syndrome; end-stage renal disease (ESRD); muscle fatigue or inflammation and dermal conditions; inflammatory conditions caused by bacterial infection or viral infection; tumors or cancers (e.g., soft tissue or solid tumors), such as leukemia (e.g., Hodgkin's lymphoma), glioblastoma, astrocytoma or lymphoma; and transplant rejection.

Linear antibodies: As used herein, the term “linear antibodies” refers to these antibodies including a pair of tandem Fv segments (V_{H}-C_{H1}-V_{H}-C_{H1}) which form a pair of antigen binding regions. Linear antibodies can be bi-specific or monospecific. Details are described in Zapata et al Protein Eng. 8(10):1057-1062 (1995).

Mammal: As used herein, the term “mammal” (also referred to as “mammalian subject,” “individual” or “patient”) includes a human or a non-human mammalian subject including, but not limited to, a bovine, cat, dog, ferret, gerbil, goat, guinea pig, hamster, horse, mouse, nonhuman primate, pig, rabbit, rat, and sheep.

Modulator: As used herein, the term “modulator” refers to a compound that alters or elicits an activity. For example, the presence of a modulator may result in an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in a activity that does not occur in the absence of the modulator.

Single-chain Fv (ScFv): As used herein, “single-chain Fv” or “ScFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the ScFv to form the desired structure for antigen binding. See, Pluckthun in The Pharmacology of Monoclonal Antibodies, vol. 113, Rosenburg and Moore eds. Springer-Verlag, New York, pp. 269-315 (1994).

Single domain antibodies: As used herein, “single domain antibodies” can include antibodies whose complementary determining regions are part of a single domain polypeptide. Examples include, but are not limited to, heavy chain antibodies, antibodies naturally devoid of light chains, single domain antibodies derived from conventional 4-chain antibodies, Single domain antibodies may be any of the art, or any future single domain antibodies. Single domain antibodies may be derived from any species including, but not limited to mouse, human, camel, llama, fish, shark, goat, rabbit, and bovine.

Single domain binding proteins: As used herein, “single domain binding proteins” can be any single domain binding scaffold that binds to an antigen, protein or peptide. Single domain binding proteins can include natural, synthetic or genetically engineered protein scaffold that act like an antibody by binding to specific antigen to form a complex and elicit a biological response (e.g., agonize or antagonize a particular biological activity). Single domain binding proteins may be derived from naturally occurring antibodies or synthetically engineered. Single domain binding proteins may be any of the art or any future single domain binding proteins, and may be derived from any species including, but not limited to mouse, human, camel, llama, fish, shark, goat, rabbit, and bovine. In some embodiments of the invention, a single domain binding protein scaffold can be derived from a variable region of the immunoglobulin found in fish, such as, for example, that which is derived from the immunoglobulin isotype known as Novel Antigen Receptor (NAR) found in the serum of shark. Methods of producing single domain binding scaffolds derived from a variable region of NAR (“IgNARs”) are described in WO 03/014161 and Streltsov (2005) Protein Sci. 14:2901-2909. In other embodiments, a single domain binding protein is a naturally occurring single domain binding protein known as a heavy chain antibody devoid of light chains. Such single domain binding proteins are disclosed in WO 9404678, for example. For clarity reasons, the variable domain derived from a heavy chain antibody naturally devoid of light chain is known herein as a VHH or “nanobody” to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain, and such VHHs are within the scope of the invention.

Small Modular ImmunoPharmaceuticals (“SMIP™”): As used herein, the term “Small Modular ImmunoPharmaceuticals (“SMIP™”), typically refers to binding domain-immunoglobulin fusion proteins including a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al. for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen.

Stringent conditions: As used herein, the term “stringent condition(s)” (also referred to as “high stringency”) refers to conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like. Exemplary stringent conditions may include low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

Substantially complementary: As used herein, the term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions.

Tetrabodies: As used herein, the term “tetrabodies” refers to a complex including four antigen-binding domains, where the four antigen-binding domains may be directed towards the same or different epitopes. Tetrabodies are constructed with the amino acid terminus of a VL or VH domain, i.e., without any linker sequence. A tetrabody can be combination of three single chain antibodies.

Therapeutically effective amount: As used herein, the term “therapeutically effective amount” of a pharmaceutical agent or combination of agents is intended to refer to an amount of agent(s) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent or composition effective to treat, ameliorate, or prevent a desired disease or condition, or to exhibit a detectable therapeutic or preventative effect. The effect can be detected by, for example, chemical markers, antigen levels, or changes in physiological indicators such as airway resistance. Therapeutic effects also include reduction in physical symptoms, such as decreased bronchoconstriction or decreased airway resistance, and can include subjective improvements in well-being noted by the subjects or their caregivers. A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific pharmaceutical agent employed; the duration of the treatment; and like factors as is well known in the medical arts.

Treatment: As used herein, the term “treatment” (also “treat” or “treating”) refers to any administration of a pharmaceutical agent that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

Triabodies: As used herein, the term “triabodies” refers to the combination of three single chain antibodies. Triabodies is also known as “trivalent trimers.” Triabodies are constructed with the amino acid terminus of a V_{L} or V_{H} domain, i.e., without any linker sequence. A triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bi-specific or trispecific.

### BRIEF DESCRIPTION OF THE DRAWINGS

The drawings are for illustration purposes only, not for limitation.

FIG. 1 depicts exemplary data illustrating that OA/OA-treatment induces inflammatory cell infiltration and increased contractility in trachea. TSM contractility (A) and counts of total cells, lymphocytes and eosinophils from the BAL (B-D) were examined in PBS/PBS-, OA/PBS- and OA/OA-treated mice. Results are expressed as Mean±SE (N=6). *: P<0.05 or **: P<0.01 OA/OA- vs either PBS/PBS- or OA/PBS-treated mice.

FIG. 2 depicts exemplary data illustrating FIZZ1 mRNA and protein expression in the OA model. The FIZZ1 mRNA expression in trachea was assayed by transcriptional profiling. FIZZ1 levels are represented as the fold change (Fc) of mRNA from tracheas from mice treated with PBS/PBS, OA/PBS and OA/OA vs naïve animals. In addition, the level of FIZZ1 protein in the BAL from PBS/PBS-, OA/PBS- and OA/OA-treated mice was determined using an anti-FIZZ1 antibody by Western Blot.

FIG. 3 depicts exemplary data illustrating that recombinant FIZZ1 (rFIZZ1) or mechanical removal results in the loss of the luminal epithelial layer. Histological examination of airway structure and the status of the airway epithelial layer was performed on frozen sections (either whole or sectional) of fresh or cultured tracheal rings treated with PBS and 100 nM FIZZ1 under a light microscope at a magnification of ×4.0 and ×20.

FIG. 4 depicts exemplary data illustrating that recombinant FIZZ1 increases CCh-generated force. CCh-generated force in PBS-, LPS- and rFIZZ1-treated trachea was recorded as original tracings (upper panel). Cumulative concentration-response curves of isometric tension to CCh stimulation were completed in PBS- and native rFIZZ1-treated (10 nM or 100 nM) trachea. MLCK and MLC-20 protein expression levels were measured by Western blot analysis in relation to the expression level of β-actin in the same tissue (A insert). The CCh-dose response curves were also performed in trachea treated with 0.1 ng/ml of LPS, 100 nM native FIZZ1 and 100 nM heat-inactivated FIZZ1 (B). Tension measurements from groups (N=6) are expressed as Mean±SEM. *: P<0.05 rFIZZ1 vs PBS- or heat-treated FIZZ1 groups.

FIG. 5 depicts exemplary data illustrating that rFIZZ1 does not affect ISO-induced relaxation. Cumulative concentration-response curves of tension to ISO stimulation were measured in the tracheal rings treated with rFIZZ1 (10 nM and 100 nM) and PBS. Tension measurements from the groups (N=6) are expressed as Mean±SEM. *: P<0.05 rFIZZ1- vs PBS-treated tracheas.

FIG. 6 depicts exemplary data from experiments measuring the force response of TSM and the infiltration of BAL cells in rFIZZ1-challenged mice. TSM force response (A) and counts of BAL cells (B) were examined in mice receiving an intranasal PBS, LPS (0.1 ng/ml) or rFIZZ1 (100 nM) dose once per day for 5 days. Results are expressed as Mean±SEM (N=5). *, **: P<0.05 vs either PBS- or LPS-treated mice.

FIG. 7 depicts exemplary data from experiments analyzing the effect of rFIZZ1 on MTEC and trachea without intact epithelium. MTEC apoptosis index (A) and nitrite concentrations (B) were examined in supernatants from treated MTEC. Cumulative dose-response curves of isometric tension to CCh stimulation were measured in trachea with epithelium, EP(+) and those with mechanically removed epithelium, EP(−), treated with PBS or FIZZ1. Tension measurements for the groups (N=8-19) are expressed as Mean±SEM. *: P<0.05, **: P<0.01 and #: P<0.07.

FIG. 8 depicts exemplary data illustrating that phosphorylation of c-Raf/ERK1/2/p38 MAPK is increased in rFIZZ1-treated trachea. Expression levels of α-actin and various G proteins (A), as well as proteins involved in the MAPK pathway such as c-Raf, phospho-c-Raf, ERK1/2, phospho-ERK1/2, p38 MAPK and phospho-p38 MAPK (B,C) were examined by Western blot in either 100 nM rFIZZ1- or PBS-treated trachea. Individual phospho-proteins were measured at the indicated time points in reference to the expression level of β-actin in the same sample (B). The expression level of total protein and the phospho-protein was determined after 24 hours incubation with either PBS or rFIZZ1 (C). Quantitation of the intensity of the protein bands from the 24 hour culture was performed (D-F). *, **, P<0.05 or 0.01 vs PBS (n=3), respectively.

### DETAILED DESCRIPTION OF THE INVENTION

The present invention provides methods for treating airway hyperresponsiveness and other inflammation diseases, disorders or conditions, in particular, those associated with digestive, pulmonary or reproductive systems, by reducing FIZZ1 activity. The present invention also provides methods for identifying modulators of airway inflammation and hyperresponsiveness and modulators of FIZZ1 and the uses thereof. In addition, the present invention provides compositions and methods for enhancing immune responses using FIZZ1 proteins, variants or fragments thereof.

The present invention is based on the discovery that FIZZ1 is a new inflammatory mediator. In particular, the present inventors found that the level of FIZZ1 mRNA and protein was upregulated in tissues from ovalbumin (OA)-treated mice and that FIZZ1 modulates the functional response of tracheal smooth muscle (TSM). For example, as described in the examples section, the tracheal rings from OA-treated mice had a significant enhancement in carbachol (CCh)-generated force with a large infiltration of cells into the bronchoalveolar lavage fluid (BAL). In association with this increased force generation, FIZZ1 mRNA expression was induced in the trachea and the expression of FIZZ1 protein was increased in the BAL from OA-treated mice compared to PBS-treated animals. Histologically, the airway epithelial layer became thinner and discontinuous in FIZZ1 (e.g., 100 nM)-treated trachea. The inventors further observed that, with the mechanical removal of the epithelium, the trachea displayed an increase in the force response of the TSM, whereas the response was more pronounced in the denuded trachea treated with FIZZ1. Additionally, an increased expression of myosin light chain kinase (MLCK), myosin light chain (MLC)-20 as well as such signal transduction molecules as phospho-c-Raf, phospho-ERK1/2 and phospho-p38 MAP kinase (MAPK) were detected in FIZZ1-treated trachea. Without wishing to be bound by any theories, it is contemplated that FIZZ1 potentiates the force development in TSM through impairing the airway epithelium and mediating MLC-20 phosphorylation via a c-Raf-ERK1/2-p38 MAPK pathway in the intact contracted muscle.

Thus, the present invention provides methods and compositions for treating inflammatory diseases, disorders, and conditions by inhibiting FIZZ1 activity using, for example, anti-FIZZ1 antibodies and anti-sense RNAs. The invention also provides methods and compositions for enhancing an immune response based on FIZZ1 proteins.

Various aspects of the invention are described in detail in the following sections. The use of sections is not meant to limit the invention. Each section can apply to any aspect of the invention. In this application, the use of “or” means “and/or” unless stated otherwise.

### FIZZ1

As used herein, the terms “FIZZ1 polypeptide,” “FIZZ1 protein” and “FIZZ1” (used inter-changeably) encompass both naturally-occurring FIZZ1 sequences and FIZZ1 variants (which are further defined herein). A FIZZ1 polypeptide suitable for the invention may be isolated from a variety of sources, such as from human or non-human (e.g., mouse) tissues, or prepared by recombinant or synthetic methods.

As used herein, a “naturally-occurring FIZZ1” includes a polypeptide having the same amino acid sequence as a FIZZ1 polypeptide derived from nature sources. Such naturally-occurring FIZZ1 can be isolated from nature or can be produced by recombinant or synthetic means. The term “naturally-occurring FIZZ1” also encompasses naturally-occurring truncated forms of the FIZZ1 proteins, naturally-occurring variant forms (e.g., alternatively spliced forms) and naturally-occurring allelic variants.

As non-limiting examples, the nucleotide sequence of murine FIZZ1 is shown in Table 1. The start and stop codons are underlined. The amino acid sequence of murine FIZZ1 is shown in Table 2.

TABLE 1

Murine FIZZ1 (mFIZZ1) nucleotide sequence
(GenBank Accession # NM_020509) (SEQ ID NO: 1)

```
    1 ggtacctagg tcagcaatcc catggcgtat aaaagcatct catctggcca ggtcctggaa
  61 cctttcctga gattctgccc caggatgcca actttgaata ggatgaagac tacaacttgt
121 tcccttctca tctgcatctc cctgctccag ctgatggtcc cagtgaatac tgatgagacc
181 atagagatta tcgtggagaa taaggtcaag gaacttcttg ccaatccagc taactatccc
241 tccactgtaa cgaagactct ctcttgcact agtgtcaaga ctatgaacag atgggcctcc
301 tgccctgctg ggatgactgc tactgggtgt gcttgtggct ttgcctgtgg atcttgggag
361 atccagagtg gagatacttg caactgcctg tgcttactcg ttgactggac cactgcccgc
421 tgctgccaac tgtcctaaga atgaagaggt ggagaaccca gctttgatat gatgaatcta
481 acaaaaactg cagtctcaat ttggaaatct gactcatgtg cctttaaatg tgttcatatt
541 gcccatttac cctgcttctt gaaatgcttc ttgaaaaata aagacaaatt tgcatgtg
```

TABLE 2

mFIZZ1 polypeptide sequence
(Dayhoff Accession # P_Y32328)

MKTTTCSLLICISLLQLMVPVNTDETIEIIVENKVKELLANIPANYPSTV
TKTLSCTSVKTMNRWASCPAGMTATGCACGFACGSWEIQSGDTCNCLCLL
V (SEQ ID NO: 2)

As other non-limiting examples, the nucleotide sequence of human FIZZ1 is shown in Table 3. The start and stop codons are underlined. The amino acid sequence of human FIZZ1 is shown in Table 4.

TABLE 3

Human FIZZ1 (hFIZZ1) nucleotide sequence
(GenBank Accession # NM_032579) (SEQ ID NO: 3)

```
    1 ccacgttgtc ttctttcctt caccaccacc caggagctca gagatctaag ctgctttcca
  61 tcttttctcc cagccccagg acactgactc tgtacaggat ggggccgtcc tcttgcctcc
121 ttctcatcct aatccccctt ctccagctga tcaacccggg gagtactcag tgttccttag
181 actccgttat ggataagaag atcaaggatg ttctcaacag tctagagtac agtccctctc
241 ctataagcaa gaagctctcg tgtgctagtg tcaaaagcca aggcagaccg tcctcctgcc
301 ctgctgggat ggctgtcact ggctgtgctt gtggctatgg ctgtggttcg tgggatgttc
361 agctggaaac cacctgccac tgccagtgca gtgtggtgga ctggaccact gcccgctgct
421 gccacctgac ctgacaggga ggaggctgag aactcagttt tgtgaccatg acagtaatga
481 aaccagggtc ccaaccaaga aatctaactc aaacgtccca cttcatttgt tccattcctg
541 attcttgggt aataaagaca aactttgtac ctcaaaaaaa aaaaaaaaaa aaaa
```

TABLE 4

hFIZZ1 polypeptide sequence
(the protein sequence accession number: NP_115968)

MGPSSCLLLILIPLLQLINPGSTQCSLDSVMDKKKIKDVLNSLEYSPSPI
SKKLSCASVKSQGRPSSCPAGMAVTGCACGYGCGSWDVQLETTCHCQCSV
VDWTTARCCHLT (SEQ ID NO: 4)

The use of the same suffix in a murine and human protein does not necessarily mean, however, that the human protein is the human homologue of the murine protein. It is possible, and contemplated, that further murine and human FIZZ proteins exist and can be identified, and the human proteins disclosed herein may be the homologues of other murine FIZZ proteins not yet identified.

A FIZZ1 polynucleotide sequence suitable for the invention includes a polynucleotide sequence provided in Tables 1 or 3, or a fragment thereof. The invention can also use a mutant or variant FIZZ1 sequence whose bases may be changed from the corresponding base shown in Tables 1 and 3 while still encoding a protein that maintains the activities and physiological functions of FIZZ1 protein, or a fragment of such a nucleic acid. A FIZZ1 polynucleotide further includes a nucleic acid molecule whose sequences are complementary to the above-described sequences, including complementary nucleic acid fragments. The polynucleotides or nucleic acids suitable for the invention can have chemical modifications. Such modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In some embodiments, up to 20% or more of the bases may be so changed (e.g., up to 1%, 2%, 3%, 4%, 5%, 6%, 7%, 8%, 9%, 10%, 11%, 12%, 13%, 14%, 15%, 16%, 17%, 18%, 195, 20% or more bases may be changed).

A FIZZ1 polynucleotide sequence suitable for the invention also includes a FIZZ1 polynucleotide variant having 70-100%, including 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%, sequence identity to the polynucleotide sequences shown in Tables 1 and 3 (SEQ ID NOs: 1 and 3, respectively). In particular, a FIZZ1 polynucleotide variant encodes a functional or active FIZZ1 protein as defined herein.

A FIZZ1 polypeptide suitable for the invention includes a polypeptide sequence provided in Tables 2 (SEQ ID NO:2) or 4 (SEQ ID NO:4), or fragments thereof. A FIZZ1 polypeptide suitable for the invention also includes a FIZZ1 mutant or variant protein. A suitable FIZZ1 mutant or variant may contain residues that differ from the corresponding residues shown in Tables 2 and 4, while still encoding a protein that maintains its biological activities and physiological functions, or a functional fragment thereof. In some embodiments, up to 30% or more of the residues may be so changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a FIZZ1 polypeptide suitable for the invention includes a polypeptide having an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NOs:2 or 4. In some embodiments, a suitable FIZZ1 polypeptide variant encodes a functional or active FIZZ1 protein as defined herein.

As used herein, an “active” or “functional” FIZZ1 protein (used inter-changeably) refers to a FIZZ1 polypeptide or FIZZ1 polypeptide fragment that retains a biological and/or an immunological activity similar, but not necessarily identical, to an activity of a naturally-occurring (wild-type) FIZZ1 polypeptide, including mature forms. A particular biological assay, with or without dose dependency, can be used to determine FIZZ1 activity. For example, in vitro assays as described in the Examples below can be used to determine FIZZ1 activity. As used herein, immunological activity refers to the ability to induce the production of an antibody against an antigenic epitope possessed by a native FIZZ1; biological activity refers to a function, either inhibitory or stimulatory, caused by a native FIZZ1 that excludes immunological activity.

“Percent (%) nucleic acid sequence identity” with respect to the FIZZ1 sequences identified herein is defined as the percentage of nucleotides in a candidate sequence that are identical with the nucleotides in the FIZZ1 sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology, 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

“Percent (%) amino acid sequence identity” with respect to the FIZZ1 sequences identified herein is defined as the percentage of amino acid residues in a candidate sequence that are identical with the amino acid residues in the FIZZ1 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

FIZZ1 mutants or variants can be prepared by introducing appropriate nucleotide changes into the FIZZ1 DNA, or by synthesis of the desired FIZZ1 polypeptide. Those skilled in the art will appreciate that amino acid changes may alter post-translational processes of the FIZZ1, such as changing the number or position of glycosylation sites or altering the membrane anchoring characteristics.

Variations in the FIZZ1 sequence or in various domains of the FIZZ1 polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the FIZZ1 that results in a change in the amino acid sequence of the FIZZ as compared with a naturally-occurring sequence of FIZZ1. Optionally the variation is by substitution of at least one amino acid with any other amino acid in one or more of the domains of the FIZZ1 protein. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting variants for activity in the in vitro assays known in the art or as described in the Examples below.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the FIZZ variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

“Isolated,” when used to describe the various FIZZ1 polypeptides disclosed herein, means polypeptide that has been identified and separated and/or recovered from a component of its natural environment. In some embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the FIZZ natural environment will not be present. Ordinarily, however, isolated polypeptide will be prepared by at least one purification step.

An “isolated” FIZZ nucleic acid molecule is a nucleic acid molecule that is identified and separated from at least one contaminant nucleic acid molecule with which it is ordinarily associated in the natural source of the FIZZ nucleic acid. An isolated FIZZ nucleic acid molecule is other than in the form or setting in which it is found in nature. Isolated FIZZ nucleic acid molecules therefore are distinguished from the FIZZ nucleic acid molecule as it exists in natural cells. However, an isolated FIZZ nucleic acid molecule includes FIZZ nucleic acid molecules contained in cells that ordinarily express FIZZ where, for example, the nucleic acid molecule is in a chromosomal location different from that of natural cells.

### Methods of Decreasing FIZZ1 Activity

Methods suitable for decreasing FIZZ1 activity can be any methods that directly or indirectly inhibit, disrupt, decrease, or reduce FIZZ1 expression or protein activity. Exemplary methods include, but are not limited to, antibody therapy, binding protein therapy, siRNA therapy, antisense therapy, ribozyme therapy, aptamer therapy, or other therapies including those using small molecules.

Antibody Therapy

Anti-FIZZ1 antibodies suitable for the invention include antibodies or fragments of antibodies that bind immunospecifically to any FIZZ1 epitopes. As used herein, the term “antibodies” is intended to include immunoglobulins and fragments thereof which are specifically reactive to the designated protein or peptide, or fragments thereof. Suitable antibodies include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), proteins, and antibody fragments. As used herein, the term “antibodies” also includes intact monoclonal antibodies, polyclonal antibodies, multispecific antibodies (e.g. bi-specific antibodies) formed from at least two intact antibodies, and antibody fragments so long as they exhibit the desired biological activity.

As used herein, an “antibody fragment” includes a portion of an intact antibody, such as, for example, the antigen-binding or variable region of an antibody. Examples of antibody fragments include Fab, Fab′, F(ab′)2, and Fv fragments; single domain antibodies; diabodies; triabodies; tetrabodies; linear antibodies; single-chain antibody molecules; and multi specific antibodies formed from antibody fragments.

Exemplary forms of anti-FIZZ1 antibodies are described below.

1. Polyclonal Abs (pAbs)

Polyclonal Abs can be raised in a mammalian host (e.g., mouse, rat, rabbit, pig, monkey, horse, dog, cat), for example, by one or more injections of an immunogen and, if desired, an adjuvant. Typically, the immunogen and/or adjuvant are injected in the mammal by multiple subcutaneous or intraperitoneal injections. The immunogen may include FIZZ1 or a fusion protein. Examples of adjuvants include Freund's complete and monophosphoryl Lipid A synthetic-trehalose dicorynomycolate (MPL-TDM). To improve the immune response, an immunogen may be conjugated to a protein that is immunogenic in the host, such as keyhole limpet hemocyanin (KLH), serum albumin, bovine thyroglobulin, and soybean trypsin inhibitor. Protocols for antibody production are described by (Ausubel et al., 1987; Harlow and Lane, 1988). Alternatively, pAbs may be made in chickens, producing IgY molecules (Schade et al., 1996).

In some embodiments, anti-FIZZ1 antibodies suitable for the present invention are subhuman primate antibodies. For example, general techniques for raising therapeutically useful antibodies in baboons may be found, for example, in Goldenberg et al., international patent publication No. WO 91/11465 (1991), and in Losman et al., Int. J. Cancer 46: 310 (1990).

2. Monoclonal Abs (mAbs)

Anti-FIZZ1 mAbs may be prepared using hybridoma methods (Milstein and Cuello, 1983). Hybridoma methods include at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-FIZZ1) mAb.

A mouse, rat, guinea pig, hamster, camel, llama, shark, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are commonly used. The immunogen typically includes a FIZZ1 polypeptide or a fusion protein containing a FIZZ1 polypeptide or a fragment thereof.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used. For example, rat or mouse myeloma cell lines mat be used. To select hybridoma cells, the cells after fusion are grown in a suitable medium that contains one or more substances that inhibit the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient unfused cells while permitting hybridomas to grow.

In some embodiments, murine myeloma lines, available from the American Type Culture Collection (Manassas, Va.), are used. In some embodiments, human myeloma and mouse-human heteromyeloma cell lines are used for the production of human mAbs (Kozbor et al., 1984; Schook, 1987).

Because hybridoma cells secrete antibody extracellularly, the culture media can be assayed for the presence of mAbs directed against FIZZ1 (anti-FIZZ1 mAbs). Suitable assays that can be used to measure the binding specificity of mAbs include, but are not limited to, immunoprecipitation or in vitro binding assays, such as radio immunoassay (RIA) or enzyme-linked immunoabsorbent assay (ELISA) (Harlow and Lane, 1988; Harlow and Lane, 1999), including Scatchard analysis (Munson and Rodbard, 1980).

Anti-FIZZ1 mAb secreting hybridoma cells may be isolated as single clones by limiting dilution procedures and sub-cultured (Goding, 1996). Suitable culture media include Dulbecco's Modified Eagle's Medium, RPMI-1640, or if desired, a protein-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as protein A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-FIZZ1 mAbs can be readily isolated and sequenced using conventional procedures, e.g., using oligonucleotide probes that specifically bind to murine heavy and light antibody chain genes, to probe preferably DNA isolated from anti-FIZZ1-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig protein, to express mAbs. The isolated DNA fragments can be modified, for example, by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

### 3. Monovalent Abs

The Abs may be monovalent Abs that consequently do not cross-link with each other. For example, one method involves recombinant expression of Ig light chain and modified heavy chain. Heavy chain truncations generally at any point in the Fc region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as Fab fragments (Harlow and Lane, 1988; Harlow and Lane, 1999).

### 4. Single Domain Antibodies

The invention also contemplates the use of single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine.

### 5. Humanized and Human Abs

Anti-FIZZ1 Abs may further comprise humanized or human Abs. Humanized forms of non-human Abs are chimeric Igs, Ig chains or fragments (such as Fv, Fab, Fab′, F(ab′)2 or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In some embodiments, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fc), typically that of a human Ig (Jones et al., 1986; Presta, 1992; Riechmann et al., 1988).

Human Abs can also be produced using various techniques, including phage display libraries (Hoogenboom et al., 1991; Marks et al., 1991) and the preparation of human mAbs (Boerner et al., 1991; Reisfeld and Sell, 1985). Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (Fishwild et al., High-avidity human IgG kappa monoclonal antibodies from a novel strain of minilocus transgenic mice, Nat. Biotechnol. 1996 July; 14(7):845-51; Lonberg et al., Antigen-specific human antibodies from mice comprising four distinct genetic modifications, Nature 1994 April 28; 368(6474):856-9; Lonberg and Huszar, Human antibodies from transgenic mice, Int. Rev. Immunol. 1995; 13(1):65-93; Marks et al., By-passing immunization: building high affinity human antibodies by chain shuffling. Biotechnology (NY). 1992 July; 10(7):779-83).

6. Bi-Specific mAbs

Bi-specific Abs are monoclonal antibodies, preferably human or humanized, that have binding specificities for at least two different antigens. For example, one binding specificity is FIZZ1; the other is for any antigen of choice, preferably a cell-surface protein or receptor or receptor subunit.

Traditionally, the recombinant production of bi-specific Abs is based on the co-expression of two Ig heavy-chain/light-chain pairs, where the two heavy chains have different specificities (Milstein and Cuello, 1983). Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This mechanism increases the yield of the heterodimer over unwanted end products such as homodimers.

Bi-specific Abs can be prepared as full length Abs or antibody fragments (e.g. F(ab′)2 bi-specific Abs). One technique to generate bi-specific Abs exploits chemical linkage. Intact Abs can be proteolytically cleaved to generate F(ab′)2 fragments (Brennan et al., 1985). Fragments are reduced with a dithiol complexing agent, such as sodium arsenite, to stabilize vicinal dithiols and prevent intermolecular disulfide formation. The generated Fab′ fragments are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bi-specific antibody. The produced bi-specific Abs can be used as agents for the selective immobilization of enzymes.

Fab′ fragments may be directly recovered from E. coli and chemically coupled to form bi-specific Abs. For example, fully humanized bi-specific F(ab′)2 Abs can be produced (Shalaby et al., 1992). Each Fab′ fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun proteins are linked to the Fab′ portions of two different Abs by gene fusion. The antibody homodimers are reduced at the hinge region to form monomers and then re-oxidized to form antibody heterodimers. This method can also produce antibody homodimers. The “diabody” technology (Holliger et al., 1993) provides an alternative method to generate bi-specific antibody fragments. The fragments comprise a heavy-chain variable domain (VH) connected to a light-chain variable domain (VL) by a linker that is too short to allow pairing between the two domains on the same chain. The VH and VL domains of one fragment are forced to pair with the complementary VL and VH domains of another fragment, forming two antigen-binding sites. Another strategy for making bi-specific antibody fragments is the use of single-chain Fv (ScFv) dimers (Gruber et al., 1994). Abs with more than two valencies are also contemplated, such as tri-specific Abs (Tutt et al., 1991).

Exemplary bi-specific Abs may bind to two different epitopes on a given FIZZ1. Alternatively, cellular defense mechanisms can be restricted to a particular cell expressing the particular FIZZ1: an anti-FIZZ1 arm may be combined with an arm that binds to a leukocyte triggering molecule, such as a T-cell receptor molecule (e.g. CD2, CD3, CD28, or B7), or to Fc receptors for IgG (FcγR), such as FcγRI (CD64), FcγRII (CD32) and FcγRIII (CD16). Bi-specific Abs may also be used to target cytotoxic agents to cells that express a particular FIZZ1. These Abs possess a FIZZ1-binding arm and an arm that binds a cytotoxic agent or a radionucleotide chelator.

### 7. Heteroconjugate Abs

Heteroconjugate Abs, consisting of two covalently joined Abs, have been proposed to target immune system cells to unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

### 8. Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii proteins, Dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional protein-coupling agents, such as N-succinimidyl-3-(2-pyridyldithiol) propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1-isothiocyanatobenzyl-3-methyldiethylene triaminepentaacetic acid (MX-DTPA) is an exemplary chelating agent for conjugating radionuclide to antibody (WO 94/11026, 1994).

### 9. Effector Function Engineering

The antibody can be modified to enhance its effectiveness in treating a disease, such as inflammation. For example, cysteine residue(s) may be introduced into the Fc region, thereby allowing interchain disulfide bond formation in this region. Such homodimeric Abs may have improved internalization capability and/or increased complement-mediated cell killing and antibody-dependent cellular cytotoxicity (ADCC) (Caron et al., 1992; Shopes, 1992). Homodimeric Abs with enhanced anti-tumor activity can be prepared using hetero-bifunctional cross-linkers (Wolff et al., 1993). Alternatively, an antibody engineered with dual Fc regions may have enhanced complement lysis (Stevenson et al., 1989).

### 10. Immunoliposomes

Liposomes containing the antibody may also be formulated (U.S. Pat. No. 4,485,045, 1984; U.S. Pat. No. 4,544,545, 1985; U.S. Pat. No. 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG-PE). Such preparations are extruded through filters of defined pore size to yield liposomes with a desired diameter. Fab′ fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are contemplated.

Binding Protein Therapy

Anti-FIZZ1 binding proteins suitable for the invention include binding proteins that bind to FIZZ1 and inhibit, disrupt, decrease or reduce (e.g., antagonize) FIZZ1 expression or biological activity. FIZZ1 binding proteins can include single domain binding proteins and scaffolds. Suitable binding proteins for use in the invention can include, for example, IgNARs, VHH nanobodies and/or SMIPs.

Aptamer Therapy

Aptamers are macromolecules composed of nucleic acid (e.g., RNA, DNA) that bind tightly to a specific molecular target (e.g., a FIZZ1 protein, polypeptide or an epitope thereof). A particular aptamer may be described by a linear nucleotide sequence and is typically about 15-60 nucleotides in length. Without wishing to be bound by any theories, it is contemplated that the chain of nucleotides in an aptamer form intramolecular interactions that fold the molecule into a complex three-dimensional shape, and this three-dimensional shape allows the aptamer to bind tightly to the surface of its target molecule. Given the extraordinary diversity of molecular shapes that exist within the universe of all possible nucleotide sequences, aptamers may be obtained for a wide array of molecular targets, including proteins and small molecules. In addition to high specificity, aptamers have very high affinities for their targets (e.g., affinities in the picomolar to low nanomolar range for proteins). Aptamers are chemically stable and can be boiled or frozen without loss of activity. Because they are synthetic molecules, they are amenable to a variety of modifications, which can optimize their function for particular applications. For example, aptamers can be modified to dramatically reduce their sensitivity to degradation by enzymes in the blood for use in in vivo applications. In addition, aptamers can be modified to alter their biodistribution or plasma residence time.

Selection of aptamers that can bind FIZZ1 or a fragment thereof can be achieved through methods known in the art. For example, aptamers can be selected using the SELEX (Systematic Evolution of Ligands by Exponential Enrichment) method (Tuerk, C., and Gold, L., Science 249:505-510 (1990)). In the SELEX method, a large library of nucleic acid molecules (e.g., 10¹⁵ different molecules) is produced and/or screened with the target molecule (e.g., a FIZZ1 protein or a FIZZ1 epitope). The target molecule is allowed to incubate with the library of nucleotide sequences for a period of time. Several methods, known in the art, can then be used to physically isolate the aptamer target molecules from the unbound molecules in the mixture, which can be discarded. The aptamers with the highest affinity for the target molecule can then be purified away from the target molecule and amplified enzymatically to produce a new library of molecules that is substantially enriched for aptamers that can bind the target molecule. The enriched library can then be used to initiate a new cycle of selection, partitioning, and amplification. After 5-15 cycles of this iterative selection, partitioning and amplification process, the library is reduced to a small number of aptamers that bind tightly to the target molecule. Individual molecules in the mixture can then be isolated, their nucleotide sequences determined, and their properties with respect to binding affinity and specificity measured and compared. Isolated aptamers can then be further refined to eliminate any nucleotides that do not contribute to target binding and/or aptamer structure, thereby producing aptamers truncated to their core binding domain. S ee Jayasena, S. D. Clin. Chem. 45:1628-1650 (1999) for review of aptamer technology; the entire teachings of which are incorporated herein by reference).

Antisense and Interfering RNA Therapy

Antisense molecules are RNA or single-stranded DNA molecules with nucleotide sequences complementary to a specified mRNA. When a laboratory-prepared antisense molecule is injected into cells containing the normal mRNA transcribed by a gene under study, the antisense molecule can base-pair with the mRNA, preventing translation of the mRNA into protein. The resulting double-stranded RNA or RNA/DNA is digested by enzymes that specifically attach to such molecules. Therefore, a depletion of the mRNA occurs, blocking the translation of the gene product so that antisense molecules find uses in medicine to block the production of deleterious proteins. Methods of producing and utilizing antisense RNA are well known to those of ordinary skill in the art (see, for example, C. Lichtenstein and W. Nellen (Editors), Antisense Technology: A Practical Approach, Oxford University Press (December, 1997); S. Agrawal and S. T. Crooke, Antisense Research and Application (Handbook of Experimental Pharmacology, Volume 131), Springer Verlag (April, 1998); I. Gibson, Antisense and Ribozyme Methodology: Laboratory Companion, Chapman & Hall (June, 1997); J. N. M. Mol and A. R. Van Der Krol, Antisense Nucleic Acids and Proteins, Marcel Dekker; B. Weiss, Antisense Oligonodeoxynucleotides and Antisense RNA Novel Pharmacological and Therapeutic Agents, CRC Press (June, 1997); Stanley et al., Antisense Research and Applications, CRC Press (June, 1993); C. A. Stein and A. M. Krieg, Applied Antisense Oligonucleotide Technology (April, 1998)).

Antisense molecules and ribozymes suitable for inhibiting FIZZ1 activity can be designed based on the sequences described above and known in the art. The antisense molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding UGGT. Such DNA sequences maybe incorporated into a wide variety of vectors with suitable RNA polymerase promoters such as T7 or SP6. Alternatively, these cDNA constructs that synthesize antisense RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′O-methyl rather than phosphodiesterase linkages within the backbone of the molecule. This concept can be extended by the inclusion of nontraditional bases such as inosine, queosine, and wybutosine, as well as acetyl-, methyl-, thio-, similarly modified forms of adenine, cytidine, guanine, thymine, and uridine which are not as easily recognized by endogenous endonucleases.

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA in cells of various lineages after first undergoing processing by an RNase III-like enzyme called DICER (Bernstein et al., Nature 409:363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length typically with 1-2 nucleotide 3′ overhangs on each strand, resulting in a total length typically of between approximately 21 and 23 nucleotides.

It will also be appreciated that siRNAs can have a range of lengths, e.g., the double-stranded portion can range from 15-29 nucleotides. It will also be appreciated that the siRNA can have a blunt end or a 3′ overhang at either or both ends. If present, such 3′ overhang is often from 1-5 nucleotides in length.

siRNA has been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, T., Nat. Biotechnol., 20:446-448, May 2002. See also Yu, J., et al., Proc. Natl. Acad. Sci., 99(9), 6047-6052 (2002); Sui, G., et al., Proc. Nail. Acad. Sci., 99(8), 5515-5520 (2002); Paddison, P., et al., Genes and Dev., 16, 948-958 (2002); Brummelkamp, T. et al., Science, 296, 550-553 (2002); Miyagashi, M. and Taira, K., Nat. Biotech., 20, 497-500 (2002); Paul, C., et al., Nat. Biotech., 20, 505-508 (2002).

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

As described in these and other references, the siRNA may consist of two individual nucleic acid strands or of a single strand with a self-complementary region capable of forming a hairpin (stem-loop) structure. A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is desirable to target exons rather than introns, and it may also be particularly desirable to select sequences complementary to regions within the 3′ portion of the target transcript. Generally it is preferred to select sequences that contain approximately equimolar ratio of the different nucleotides and to avoid stretches in which a single residue is repeated multiple times.

siRNA may thus comprise RNA molecules typically having a double-stranded region approximately 19 nucleotides in length typically with 1-2 nucleotide 3′ overhangs on each strand, resulting in a total length of between approximately 21 and 23 nucleotides. As used herein, siRNA also includes various RNA structures that may be processed in vivo to generate such molecules. Such structures include RNA strands containing two complementary elements that hybridize to one another to form a stem, a loop, and optionally an overhang, preferably a 3′ overhang. Typically, the stem is approximately 19 bp long, the loop is about 1-20, preferably about 4-10, and more preferably about 6-8 nucleotides long and/or the overhang is typically about 1-20, and preferably about 2-15 nucleotides long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may, but need not, comprise a plurality of U residues, e.g., between 1 and 5 U residues.

The siRNA compounds suitable for the present invention can be designed based on the FIZZ1 sequence described above and can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res, 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

Inventive siRNAs may be comprised entirely of natural RNA nucleotides, or may instead include one or more nucleotide analogs and/or modifications as mentioned above for antisense molecules. The siRNA structure may be stabilized, for example by including nucleotide analogs at one or more free strand ends in order to reduce digestion, e.g., by exonucleases. This may also be accomplished by the inclusion. Alternatively, siRNA molecules may be generated by in vitro transcription of DNA sequences encoding the relevant molecule. Such DNA sequences may be incorporated into a wide variety of vectors with suitable RNA polymerase promoters such as T7, T3, or SP6.

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phosphodiester linkage). As is known in the art, an “unmodified ribonucleotide” has one of the bases adenine, cytosine, guanine, and uracil joined to the 1′ carbon of beta-D-ribo-furanose.

Modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res, 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman & Cedergren, Trends Biochem Sci, 17:334 (1992).

siRNA may be generated by intracellular transcription of small RNA molecules, which may be followed by intracellular processing events. For example, intracellular transcription is achieved by cloning siRNA templates into RNA polymerase III transcription units, e.g., under control of a U6 or H1 promoter. In one approach, sense and antisense strands are transcribed from individual promoters, which may be on the same construct. The promoters may be in opposite orientation so that they drive transcription from a single template, or they may direct synthesis from different templates. In a second approach siRNAs are expressed as stem-loop structures. The siRNAs of the invention may be introduced into cells by any of a variety of methods. For instance, siRNAs or vectors encoding them can be introduced into cells via conventional transformation or transfection techniques. As used herein, the terms “transformation” and “transfection” are intended to refer to a variety of aft-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation.

Vectors that direct in vivo synthesis of siRNA constitutively or inducibly can be introduced into cell lines, cells, or tissues. In certain preferred embodiments of the invention, inventive vectors are gene therapy vectors (e.g., adenoviral vectors, adeno-associated viral vectors, retroviral or lentiviral vectors, or various nonviral gene therapy vectors) appropriate for the delivery of an siRNA-expressing construct to mammalian cells, most preferably human cells. Thus the present invention includes gene therapy approaches to the treatment of diseases or clinical conditions associated with inflammation in, for example, airway (e.g., airway hyperresponsiveness), digestive, pulmonary or reproductive tract.

The invention includes methods of treating a disease or clinical condition associated with inflammation in, for example, airway, digestive, pulmonary or reproductive tract by administering siRNA compositions comprising siRNA that targets FIZZ1 or a FIZZ1 receptor. The compositions may be administered parenterally, orally, inhalationally, etc.

Typically, siRNA compositions reduce the level of the target transcript and its encoded protein by at least 2-fold, preferably at least 4-fold, more preferably at least 10-fold or more. The ability of a candidate siRNA to reduce expression of the target transcript and/or its encoded protein may readily be tested using methods well known in the art including, but not limited to, Northern blots, RT-PCR, microarray analysis in the case of the transcript, and various immunological methods such as Western blot, ELISA, immunofluorescence, etc., in the case of the encoded protein. Efficacy may be tested in appropriate animal models or in human subjects.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, e.g., using a DNA vector from which siRNA compounds in, e.g., small hairpin form (shRNA), can be transcribed directly. Numerous studies have demonstrated that while double-stranded siRNAs are very effective at mediating RNAI, short, single-stranded, hairpin-shaped RNAs can also mediate RNAI, presumably because they fold into intramolecular duplexes that are processed into double-stranded siRNAs by cellular enzymes. Sui et al., Proc Natl Acad Sci USA, 99:5515-5520 (2002); Yu et al., Proc Natl Acad Sci USA, 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet, 19:9-12 (2003); see also Xia et al., Nature Biotech., 20:1006-1010 (2002).

Additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. (Nature 411: 494-498 (2001). For example, guidelines have been compiled into “The siRNA User Guide” which is available at the website of The Rockefeller University, New York, N.Y.

### Identification of FIZZ1 Modulators

The present invention also provides methods for evaluating or identifying modulators of FIZZ1 activity or biological/physiological functions that involve FIZZ1, in particular, in connection with inflammation. In particular, the present invention provides methods (e.g., screening assays) for identifying modalities, i.e., candidate or test compounds or agents (e.g., peptides, peptidomimetics, small molecules or other drugs), that modulate FIZZ1 (e.g., stimulates or inhibits), including translation, transcription, activity, in particular, physiological activity in connection with inflammation (e.g., airway inflammation or hyperresponsiveness).

In some embodiments, high throughput screening is utilized in the search for modulators which are capable of modulate biological/physiological function of FIZZ1 (e.g., airway inflammation or airway hyperresponsiveness). The assays described below can be designed to permit rapid automated screening of large numbers of agents useful for practicing the claimed invention. For general information on high-throughput screening, see, for example, Cost-Effective Strategies for Automated and Accelerated High-Throughput Screening, IBCS Biomedical Library Series, IBC United States Conferences (February, 1996); John P. Devlin (Editor), High Throughput Screening, Marcel Kedder (1998); U.S. Pat. No. 5,763,263.

Assays can be developed based on the discovery that FIZZ1 potentiates the force development in trachea and impair the airway epithelium. One exemplary method includes the steps of: (1) providing a trachea sample; (2) culturing the trachea sample in a medium in the presence of FIZZ1; (3) providing an agent to the medium; (4) determining the histology of the trachea sample; and (5) comparing the histology result from step (4) to a control to evaluate the ability of the agent to modulate airway inflammation. In some embodiments, step (4) includes determining the histological intactness of the epithelial layer in the trachea sample. In some embodiments, the control includes the histology of a tracheal sample cultured in the medium in the absence of FIZZ1. In some embodiments, the control includes the histology of a tracheal sample cultured in the medium in the presence of FIZZ1.

Another exemplary method includes the steps of: (1) providing a trachea sample; (2) culturing the trachea sample in a medium in the presence of FIZZ1; (3) providing an agent to the medium; (4) providing carbachol to the medium; (5) determining a contractile response to carbachol of the trachea sample; and (6) comparing the contractile response to carbachol determined in step (5) to a control to evaluate the ability of the agent to modulate airway hyperresponsiveness. In some embodiments, the control includes the contractile response to carbachol of a tracheal sample cultured in the medium in the absence of FIZZ1. In some embodiments, the control includes the contractile response to carbachol of a tracheal sample cultured in the medium in the presence of FIZZ1.

Trachea samples suitable for the above assays can be derived from a mouse, a rat, a sheep, a cow, a cat, a guinea pig, or other animals. Preferably, the animals are treated with allergens (e.g., ovalbumin or lipopolysaccharide), or other antigens (e.g., Ascaris suum antigen), before the trachea sample was taken.

For example, tissue samples (e.g., trachea) may be derived from animal models that are known in the art (e.g. U.S. Pat. Nos. 6,193,957; 6,051,566; 5,080,899, 6,180,643, 6,028,208 and U.S. Pat. App. Nos. 20010000341, 20010006656). For example, U.S. Pat. No. 6,193,957 describes in detail an in vivo model (sheep) of pulmonary airflow resistance. U.S. Pat. No. 5,080,899 details an in vivo guinea pig model for studying the efficacy of orally administered drugs for the treatment of pulmonary inflammation. U.S. Publication Nos. 20010000341 and 20010006656 describe in vivo models of LPS-induced airway inflammation in mice. U.S. Pat. No. 6,028,208 describes a similar in vivo model of LPS-induced airway inflammation in hamsters.

Assays based on FIZZ1-mediated phenotypes can also be used to identify FIZZ1 modulators, in particular, FIZZ1 inhibitors. One exemplary method includes the steps of: (1) providing a plurality of trachea samples, each of which is cultured in a medium in the presence of FIZZ1; (2) providing a plurality of inhibitor candidates; (3) determining a phenotype associated with FIZZ1-mediated airway inflammation or hyperresponsiveness in each of the plurality of trachea samples; (4) comparing the phenotype determined in step (3) to a control; and (5) identifying one or more inhibitors of FIZZ1 that reduce the phenotype based on the comparison result in step (4). In some embodiments, the plurality of inhibitor candidates include a small molecule library. In some embodiments, the plurality of inhibitor candidates include an antibody library. In some embodiments, the antibody library suitable for the method of this aspect of the invention is a single chain Fv library. In some embodiments, the plurality of inhibitor candidates include an interfering RNA library. In some embodiments, the plurality of inhibitor candidates include an aptamer library (e.g., an RNA aptamer library). In some embodiments, step (3) includes determining the histology of each of the plurality of trachea samples. In some embodiments, step (3) includes determining contractile response to carbachol.

As used herein, a “small molecule” refers to a composition that has a molecular weight of less than about 5 kD and more preferably less than about 4 kD, and most preferable less than 0.6 kD. Exemplary small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds). Small molecules also include salts, esters, and other pharmaceutically acceptable forms of such compounds. Examples of methods for the synthesis of molecular libraries can be found in: Carell et al., 1994a; Carell et al., 1994b; Cho et al., 1993; DeWitt et al., 1993; Gallop et al., 1994; Zuckermann et al., 1994.

Other methods for identifying FIZZ1 modulators are well known in the art including, but not limited to, two-hybrid system, phage display, ribosome display, yeast display, other methods for assaying protein-protein interactions and computerized methods including those for rational drug designs.

### Determination of the Therapeutic Effect of FIZZ1 Modulators

Suitable in vitro or in vivo assays can be performed to determine the therapeutic effect of a particular FIZZ1 modulator and/or whether its administration is indicated for treatment of the affected tissue.

In various specific embodiments, in vitro assays may be performed with representative cell types derived from tissues involved in the patient's disorder, to determine if a given modulator exerts the desired effect upon relevant cell types. Therapeutic use of the modulators may also be tested in suitable animal model systems including, but not limited to rats, mice, chicken, cows, monkeys, rabbits, and the like, prior to testing in human subjects. The therapeutic effects of modulators can be evaluated based on their effects on inflammatory symptoms, tissue histology (e.g., histology of trachea and other vascular tissues), and other inflammatory parameters, such as, for example, neutrophil count, MPO activity, or inflammatory biomarkers known in the art or as described herein. For in vivo testing, any of the animal model system known in the art or developed in the future may be used prior to administration to human subjects.

As used herein, “inflammatory biomarkers” (also referred to as “markers associated with inflammation”) include, but are not limited to CRP, cytokines associated with inflammation, such as members of the interleukin family, including IL-1 through IL-17 that are associated with inflammation, TNF-alpha; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; leukotriene, thromboxane, and isoprostane; and myosin light chain kinase (MLCK), myosin light chain (MLC)-20 as well as signal transduction molecules such as phospho-c-Raf, phospho-ERK1/2 and phospho-p38 MAPK. As non-limiting examples, elevated levels of CRP are associated with cardiovascular diseases and disorders, infectious diseases, such as, myocarditis, cardiomyopathy, acute endocarditis, or pericarditis; SIRS; diabetes; metabolic syndrome; muscle fatigue, injury or inflammation; and systemic inflammation. By way of example but not limitation: Elevated levels of IL-6, sTNFr2 and CRP are associated with type II diabetes, muscle inflammation and ESRD; elevated levels of cellular adhesion molecules are associated with systemic inflammation; elevated levels of IL-1 and TNF-alpha are associated with IDDM and NDDM associated inflammation; elevated levels of IL-10 and IL-6 are associated with SIRS; elevated levels of neopterin are associated with SIRS; elevated levels of procalcitonin are associated with systemic inflammation. Other proteins or markers associated with inflammation include serum amyloid A protein, fibrinectin, fibrinogen, leptin, prostaglandin E2, serum procalcitonin, soluble TNF receptor 2, elevated erythrocyte sedimentation rate, and elevated white blood count, including percent and total granulocytes (polymorphonuclear leukocytes), monocytes, lymphocytes and eosinophils.

For example, modulators can be tested in a mouse AHR model. AHR is a cardinal feature of bronchial asthma with proinflammatory mediators being some of the primary initiators of this altered responsiveness. AHR measured as either lung resistance (R_{L}) in vivo or the ex vivo force response of TSM tissue, has been considered a primary indicator for the efficacy of clinical drug therapy in the treatment of asthmatic attacks. An increase in R_{L} indicates the summation of multiple components involved in the process of airway narrowing, whereas the force response of airway smooth muscle solely allows the measuring of the contractile response of the muscle to agonist. A mouse AHR model was established based on the observation that a 10-day OA challenge was able to model the abnormal functional behavior of TSM in response to CCh seen in human asthma [Matsubara et al., Am J Respir Crit. Care Med, 173:56-63 (2006)]. As discussed in the examples section, the present inventors demonstrated OA challenge effect not only a significant increase in CCh-evoked force but also a large inflammatory infiltrate into the BAL, comprised mainly of lymphocytes and eosinophils. These findings, like those seen in clinical asthma, fully demonstrate the association of AHR and airway inflammation in this animal model. Exemplary methods of using the AHR mouse model are described in the examples section.

In some embodiments, modulators can be tested in a murine model treated by lipopolysaccharide (LPS) via intranasal instillation. Bacterial LPS is a macromolecular cell surface antigen of bacteria which, when applied in vivo triggers a network of inflammatory responses. The main characteristics of this LPS-induced inflammation model include, but are not limited to, macrophage activation, tumor necrosis factor-alpha (TNF-α) production and neutrophil infiltration and activation, which are features of chronic obstructive pulmonary disease. This model causes pulmonary inflammation as an acute injury which occurs after 2 to 4 hours in the airway lumen, where all the inflammatory parameters can be assessed by bronchoalveolar lavage (BAL).

As a non-limiting example, a test modulator can be dissolved in a diluent (e.g., dimethyl sulfoxide (DMSO) at a desirable concentration. Animals (e.g., Balb/C mice) can be treated intranasally, under anaesthesia, with the test modulator at a suitable dose (e.g., 0.1-30 mg/kg) or with diluent alone and, later (e.g., 30 minutes later), with allergens (e.g., LPS or OA). The animals are typically housed in plastic cages in an air conditioned room at 24° C. Food and water are available ad libitum. Typically, three hours after intranasal administration of the allergens, the animals are sacrificed.

The trachea can be cannulated and bronchoalveolar lavage (BAL) is performed by injecting PBS into the lung via the trachea. The fluid is then immediately withdrawn and the cell suspension can be stored, e.g., on ice. Total cell count is measured and cytospin preparation is prepared. The inhibitory effect of the modulator under test on lung inflammation can be examined and determined. The details of this animal model are described in U.S. Pat. No.

As another non-limiting example, a male golden hamster is placed in an inhalation chamber and allowed to inhale LPS for a period of time (e.g., 30 min) to cause airway inflammatory. Just after the inhalation of the LPS, a test modulator is administered through intrarespiratory tract administration or orally under halothane anesthesia. Typically, after 24 hr, tracheal branches and pulmonary alveoli are washed, and the number of neutrophils in the washing is determined. Using the number of neutrophils obtained in the absence of a test compound as the control, the decreasing rates of the numbers of neutrophils are expressed in terms of percent suppression based on the control. Other tests such as the histology of the trachea samples from the modulator treated mice and the control mice are also examined and compared. Details of this animal model are described in U.S. Pat. No. 6,380,259.

### Pharmaceutical Compositions

The FIZZ1 proteins or polypeptides, anti-FIZZ1 antibodies, antisense oligonucleotides, ribozymes, interfering RNAs, or modulators of the invention and derivatives thereof (collectively, “active compound” or “active ingredient”), can be incorporated into pharmaceutical compositions. Such compositions typically further include a pharmaceutically acceptable carrier or excipient. As used herein, the term “pharmaceutically acceptable carrier or excipient” means a non-toxic, inert solid, semi-solid or liquid filler, diluent, encapsulating material or formulation auxiliary of any type. Generally, examples of such carriers or excipients include, but are not limited to, water, saline, finger's solutions, dextrose solution, and 5% human serum albumin. Liposomes and non-aqueous vehicles such as fixed oils may also be used. Supplementary active compounds can also be incorporated into the compositions.

### 1. General Considerations

A pharmaceutical composition of the invention is formulated to be compatible with its intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

### 2. Injectable Formulations

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, for example, parabens, chlorobutanol, phenol, ascorbic acid, and thimerosal, can contain microorganism contamination. Isotonic agents, for example, sugars, polyalcohols such as manitol, sorbitol, and sodium chloride can be included in the composition. Compositions that can delay absorption include agents such as aluminum monostearate and gelatin.

Sterile injectable solutions can be prepared by incorporating the active compound in the required amount in an appropriate solvent with one or a combination of ingredients as required, followed by sterilization. Generally, dispersions are prepared by incorporating the active compound into a sterile vehicle that contains a basic dispersion medium, and the other required ingredients as discussed. Sterile powders for the preparation of sterile injectable solutions, methods of preparation include vacuum drying and freeze-drying that yield a powder containing the active ingredient and any desired ingredient from a sterile solution.

### 3. Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

### 4. Compositions for Inhalation

For administration by inhalation, the compounds are delivered as an aerosol spray from a nebulizer or a pressurized container that contains a suitable propellant, e.g., a gas such as carbon dioxide.

### 5. Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

6. Other formulations

In one embodiment, the active compounds are prepared with carriers that protect the active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Liposomal suspensions can also be used as pharmaceutically acceptable carriers. These can be prepared according to methods known to those skilled in the art, such as in (Eppstein et al., U.S. Pat. No. 4,522,811, 1985).

Microcapsules can be prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (Boswell and Scribner, U.S. Pat. No. 3,773,919, 1973), copolymers of L-glutamic acid and .gamma. ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogels release proteins for shorter time periods and may be preferred.

### 7. Unit Dosage

Oral formulations or parenteral compositions in unit dosage form can be created to facilitate administration and dosage uniformity. Unit dosage form refers to physically discrete units suited as single dosages for the subject to be treated, containing a unit dose of active compound in association with the required pharmaceutical carrier. The term “unit dose”, as used herein, refers to a discrete administration of a pharmaceutical composition, typically in the context of a dosing regiment. The specification for the unit dosage forms of the invention are dictated by, and directly dependent on, the unique characteristics of the active compound and the particular desired therapeutic effect, and the inherent limitations of compounding the active compound.

### 8. Gene Therapy Compositions

The nucleic acid molecules used in the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

### 9. Therapeutically Effective Amount

Typically, the precise therapeutically effective amount for a subject will depend upon the subject's size, weight, and health, the nature and extent of the condition affecting the subject, and the therapeutics or combination of therapeutics selected for administration, as well as variables such as liver and kidney function that affect the pharmacokinetics of administered therapeutics. However, the effective amount for a given situation can be determined by routine experimentation and is within the judgment of the clinician.

In general, in the treatment or prevention of inflammation conditions which require FIZZ1 modulation, a therapeutically effective amount is about 0.01 to 500 mg per kg patient body weight per day which can be administered in single or multiple doses. For example, a therapeutically effective amount may be about 0.1 to about 250 mg/kg per day, about 0.5 to about 100 mg/kg per day, about 0.01 to 250 mg/kg per day, about 0.05 to 100 mg/kg per day, about 0.1 to 50 mg/kg per day, about 0.05 to 0.5 mg/kg per day, about 0.5 to 5 mg/kg per day, or about 5 to 50 mg/kg per day. For oral administration, the compositions are typically provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The pharmaceutical composition and method of the present invention may further comprise other therapeutically active compounds that are usually applied in the treatment of the above-mentioned pathological conditions.

### 10. Vaccine Formulations

In some embodiments, a pharmaceutical composition of the invention can be formulated as a vaccine composition. For example, it is contemplated that FIZZ1 proteins, or variants or fragments thereof, can be used to enhance an inadequate immune response. Thus, a vaccine containing FIZZ1 proteins, or variants or fragments thereof can be formulated for in vivo administration to the host.

In some embodiments, the vaccine compositions of the invention may further include one or more adjuvants. Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes.

The adjuvant may also be selected to be a preferential inducer of a TH1 type of response to aid the cell mediated branch of the immune response.

High levels of Th1-type cytokines tend to favor the induction of cell mediated immune responses to a given antigen, whilst high levels of Th2-type cytokines tend to favor the induction of humoral immune responses to the antigen.

Suitable adjuvant systems which promote a predominantly Th1 response include, monophosphoryl lipid A or a derivative thereof, particularly 3-de-O-acylated monophosphoryl lipid A, and a combination of monophosphoryl lipid A, preferably 3-de-O-acylated monophosphoryl lipid A (3D-MPL) together with an aluminium salt. An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210. The vaccine may additionally comprise a saponin, more preferably QS21. The formulation may also comprise an oil in water emulsion and tocopherol (WO 95/17210). Unmethylated CpG containing oligonucleotides (WO 96/02555) are also preferential inducers of a TH1 response and are suitable for use in the present invention.

The present invention also provides a method for producing a vaccine formulation comprising the step of mixing the components of the vaccine together with a pharmaceutically acceptable excipient.

The present invention, thus generally described, will be understood more readily by reference to the following examples, which are provided by way of illustration and are not intended to be limiting of the present invention.

### EXAMPLES

### Example 1

### Preparation of Animals and Tracheas

Specific pathogen-free male BALB/C mice (5 weeks old) were used in these experiments. All of the experimental animals were housed at Wyeth Research Corporation under pathogen-free conditions for the duration of the experiments. Food and water were provided ad libitu. All studies were conducted in accordance with the National Institutes of Health Guide for the Care and Use of Laboratory Animals as well as following guidelines from, and with the approval of, the Institutional Animal Care and Use Committee of Wyeth Research Corporation.

Animals were divided into three groups including phosphate buffered saline (PBS)-sensitized and -challenged (PBS/PBS), OA-sensitized and PBS-challenged (OA/PBS), and OA-sensitized and -challenged mice (OA/OA). Mice were intraperitoneally injected with an equivalent volume (100 μL) of PBS or OA (20 μg) with 2.25 mg Al(OH)₃ in PBS on day 0 and 14. From day 25 to 34, mice were challenged with an aerosol of PBS or OA (5% in PBS) for 30 min once a day for 10 consecutive days.

Experimental animals were sacrificed by CO₂ asphyxiation. Tracheas were surgically excised and cleaned of adherent connective tissue. Each trachea was sectioned into rings 3-4 mm in length and cultured in DMEM containing 1.0 M HEPES, 1.0 M NaOH, 5% of heat-inactivated FBS (Hy-Clone, Logan, Utah), 0.2M glutamine, 1.0 M CaCl₂, 2.5 μg/ml fungizone, 5 μg/mL insulin, 100 U/mL penicillin and 100 μg/mL streptomycin) (DMEM-5%) for 24 hours. For in vitro experiments, the tracheal rings were cultured in DMEM in the absence and presence of either 10 or 100 nM recombinant FIZZ1 (Leinco Technologies, USA) for 24 hours.

For some experiments, ex vivo TSM tensions of fresh tracheal rings and BAL cell counts were measured at 24 hours after the last treatment of mice receiving an intranasal dose of either PBS, 0.1 ng/m: LPS or 100 nM rFIZZ1 (once a day×5 days).

### Example 2

### Cell Counts and Protein Preparation

PBS- and OA-treated mice were sacrificed and their airways lavaged once with 1.0 mL of PBS via tracheal cannulation. An equal volume of BAL from each mouse was collected and centrifuged (1200 rpm, 5 min). Total BAL cells were counted using a hemocytometer. BAL cells (3.0×10⁻⁴ cells) collected from each sample were applied to a glass slide using a cytospin (800 rpm, 8 min) and then the slide was stained with Hema 3 Stain Set (Fisher Scientific) for the differential count of cells. The relative proportion of different cells counted from 300 cells/slide was factored to the number of total BAL cells collected in each group.

PBS- and FIZZ1-treated trachea were cultured in DMEM overnight for Western blot analysis. Treated and untreated trachea were harvested and homogenized separately in lysis buffer containing 20 mM MOSP, 2.0 mM EGTA, 5.0 mM EDTA, 30 mM sodium fluoride, 40 mM β-glycerophosphate, 20 mM sodium orthovanadate, 1.0 mM phenylmethylsulfonyl-fluoride, 3.0 mM benzamidine, 5 μM pepstatin A, 10 μM leupeptin and 0.5% Triton X-100 at pH 7.2 (KINEXUS, Canada). Tissue supernatants were centrifuged (15,000×g) for 60 min at 4° C. and protein concentrations in the cleared supernatant of homogenized trachea and BAL were examined by bicinchoninic acid assay (BCA) (Pierce Biotechnology, Rockford, Ill.). Absorbance of total protein from each group was measured spectrophotometrically at an optical density of 562 nm and different concentrations of bovine serum albumin (BSA) were applied as a standard curve. Proteins concentrations (μg/ml) were quantified with the standard curve of BSA using BCA assay. Samples were stored at −70° C. until use.

### Example 3

### Examination of Airway Epithelium

Histological examination of the structure of the airway and the status of the airway epithelial layer was performed in whole/sectional fresh trachea and as well as trachea cultured overnight with and without 100 nM FIZZ1. To understand the role of airway epithelium in regulating contractility of TSM, we measured the contractile response of tracheal rings after the epithelium was mechanically removed. Briefly, airway epithelial cells were removed by gently rubbing the intraluminal surface with polyethylene tubing (Becton Dickinson & Company, MD USA) connected to a Precision Glide needle (30G1/2) followed by perfusion with 1.0 ml air bubbles and then 1.0 ml K-H solution (re). All of the experimental tracheas were stained with H & E solution (CAT hematoxylin, Edgar Degas Eosin Working Solution, Biocare Medical, Concord, Calif.) and photographed under a light microscope at ×4.0 and ×20 magnification. Tracheal morphometric analysis was performed using a computer-based image analysis system consisting of a Nikon Eclipse E800 microscope (Melville, N.Y. USA) with a SPOT RT Slider camera (Diagnostic Instruments, Inc., Sterling Heights, Mich. USA).

### Example 4

### Mouse Tracheal Epithelium Cell (MTEC) Culture and Assay

MTEC culture was performed by following the protocol of You et al. (“Growth and differentiation of mouse tracheal epithelial cells: selection of a proliferative population,” Am J Physiol Lung Cell Mol Physiol, 2002, 283:L 1315-1321) with minor modification. Briefly, tracheas were incubated in 1.5 mg/mL pronase (Roche Molecular Biochemicals) for 18 h at 4° C. Cells were treated with 0.5 mg/mL crude pancreatic DNase I (Sigma-Aldrich) on ice for 5 min. After incubation in tissue culture plates for 3-4 h in 5% CO₂ at 37° C., nonadherent cells were incubated in a plate coated with type I rat tail collagen (BD Biosciences) in modified BEBM (Lonza, Md. USA) containing 10 μg/ml insulin, 5 μg/ml transferrin, 25 ng/ml epidermal growth factor, 5 μg/ml epinephrine and 30 μg/ml bovine pituitary extract, 0.5 nM Hydrocortisone, 25 ng/ml hEGF, 15 nM Triiodothyronine, 0.25 μg/ml Gentamicin/amphotericin-B and 0.01 μM retinoic acid in 5% CO₂ at 37° C. MTEC were seeded on polycarbonate semipermeable membrane (0.4 μM pore size, Corning, N.Y.) and media was removed from upper chamber to establish an air-liquid interface, lower chambers only were provided with BEBM/DMEM (1:1, v/v) containing 7.5 μL retinoic acid and 750 μL BSA in presence and absence of LPS and rFIZZ1.

Apoptotic MTEC death was examined using Cell Death Detection ELISAplus (Roche) according to the manufacturer's instructions and calculated as an index of a fold change over a control. This assay is based on the sandwich-enzyme-immunoassay principle using mouse monoclonal antibodies directed against histone-associated DNA fragments. Quantitation of histone-associated-DNA-fragments in supernatants of MTEC cultures (5×10⁴/mL) treated with PBS, 0.1 ng/mL LPS or 100 nM rFIZZ1 was performed at an absorbance of 405-490 nm.

Nitric oxide (NO) was examined by measuring an end product, nitrite, using the Griess reaction (Xu et al., “Arginase and autoimmune inflammation in the central nervous system,” Immunology, 2003; 110:141-148). Briefly, aliquots (50 μL) of supernatants from treated MTEC were mixed with 50 μL Griess reagent (Bio-Rad, Hercules, Calif.) at room temperature for 10 min. Absorbance was read at 540 nm in an automated microplate reader. Nitrite concentrations were calibrated using a standard curve of sodium nitrite prepared as 200, 100, 50, 25, 12.5, 3.125 and 0 (μM).

### Example 5

### TSM Force Measurement System

Trachea were supported longitudinally by a plexiglas rod with a stainless steel pin into the base of a double-jacketed, glass organ bath filled with 10 mL of Krebs-Henseleit (K-H) solution (37° C.) of the following composition: 118 mM NaCl; 4.7 mM KCl; 1.2 mM KH₂PO₄; 11.1 mM Dextrose; 1.2 mM MgSO₄; 2.8 mM CaCl₂; and 25 mM NaHCO₃. The solution was maintained at a pH of 7.40-7.45 and continuously gassed with a mixture of 5% CO₂ and 95% atmosphere for the duration of each experiment. The upper support was attached by a loop of silk thread to a FT03 isometric transducer (BIOPAC Systems, Inc., Goleta, Ca) by which changes in the tension of the TSM were measured, and concentration-response curves were synchronously recorded with a MP 150WS system (BIOPAC Systems, Inc., Goleta, Ca) and displayed on a Macintosh computer. Initial tensions of TSM were set at approximately 0.5 g and maintained for 1 hour. Agonists were given after a steady state of tension had been reached.

### Example 6

### Pharmacodynamic Studies

[CCh]-response curves at the doses ranging from 3×10⁻⁸ to 10⁻⁵ M were completed in tracheal rings in absence and presence of either FIZZ1 or LPS (0.1 ng/mL). Concentrations of agonist were increased only when force responses to the previous concentration had stabilized. To examine TSM relaxant responses to isoprenaline (ISO), tracheal rings were first contracted by an addition of 1.0 μM CCh (Sigma, USA). Once the contraction had stabilized, ISO (Sigma, USA) was introduced into each bath at increasing concentrations (3×10⁻⁸-10⁻⁵ M). 200 μM papaverine (a phosphodiesterase inhibitor), producing complete relaxation of the trachea, was added at the end of the experiment to evaluate whether maximum relaxation was achieved with the highest concentrations of this ISO. In an additional experiment, the effect of FIZZ1 protein on the CCh-mediated force response was verified using 100 nM heat-inactivated FIZZ1 (natural and recombinant FIZZ1 solution heated at 70° C. for 60 min). Their dose-response curves were obtained as above. Fresh drug solutions such as CCh and ISO were made up on the day of the experiment. Doses of the above agents refer to the final bath concentration.

### Example 7

### Protein Gel Electrophoresis and Quantifying Western Blots

Protein expression levels of were examined using Western blot analysis. Briefly, aliquots (100 μg/well) of BAL supernatant and tracheal lysate were loaded onto 4-20% SDS-PAGE gel in an equal volume. Size-fractionated proteins were transferred to nitrocellulose membrane and then blocked with 5% nonfat dried milk in TBS at room temperature for 60 min. The membrane was individually incubated with primary antibodies to either FIZZ1 (Rabbit anti-mouse FIZZ1, Antigenix America Inc., USA), MLCK, MLC-20, α-actin, Giα1,2, Ggα11, β-actin (Sigma, USA), Gα12/13 (Santa Cruz Biotechnology, Inc., USA), c-Raf, phospho-c-Raf, ERK1/2, phospho-ERK1/2, p38 MAPK or phospho-p38 MAPK (Cell Signaling, Inc., USA) at 4° C. overnight, washed three times with TBS and then incubated with peroxidase-conjugated secondary antibodies for another 60 min. The blot was washed 3 times with TBS and a mixture of Western Blotting Detection Reagent I and II (GE Healthcare Life Sciences, Piscataway, N.J.) was poured on the membrane with gentle agitation for 1 min at room temperature. Immunoreactive bands were detected by chemiluminesence. Protein expression levels were evaluated in relative to expression of β-actin in the same tissue. Quantification of Western blots for phosphorylated signaling proteins was performed using ImageJ and relative band intensity was calculated as % of the intensity of the β-actin protein band.

### Example 8

### RNA Isolation, Purification Amplification, Labeling and Hybridization for Gene Expression Analysis

Three (3) groups of mice were analyzed using PBS/PBS, OA/PBS and OA/OA, with 18 animals per group. Tracheal rings from 6 animals per group were combined as replicates, to produce total RNA. Total RNA was extracted using a tissue homogenizer and Qiagen lysis buffer and purification of RNA was performed with Qiagen RNeasy minicolumns. RNA was quantified using the Nanoprop ND-1000 spectrophotometer. The yield of total RNA per replicate varied from 0.6 μg to 2.0 μg. 45 ng of total RNA was amplified and biotin-labeled with Nugen's Ovation System, according to the manufacturer's instructions (NuGEN Technologies, Inc., San Carlos, Calif.). The Ovation kit utilizes the Ribo-SPIA process to linearly amplify and label, limiting amounts mRNA in a three-step process resulting in microgram quantities (Kum et al, “Novel Isothermal, Linear Nucleic Acid Amplification Systems for Highly Multiplexed Applications,” Clinical Chemistry, 2005; 51:1973-1981). Approximately 1.5 μg of purified and fragmented biotinylated cRNA, together with controls for quantitating the amount of each transcript, was hybridized to the mouse gene chip array, MOE 430_{—}2.0 (Affymetrix) for 16-18 hours. GeneChips were scanned with an Agilent GeneArray scanner. Resulting signals were normalized and quantified using Gene Logic's MAS 5.0 software.

### Example 9

### Data Analysis

At the end of each force measurement experiment, tracheas were blotted on a gauze pad and weighted. Results were calculated as milligram of tension per milligram of TSM weight (mg/mg) and expressed as an individual percentage (%) of 10 μM CCh- or 200 μM papaverine-induced tension response in PBS-treated trachea. For the control group, CCh (papaverine)-mediated responses were normalized to the mean value of the maximal responses.

Values were expressed as Mean±SE. Comparisons within groups of different contractile/relaxation agonists (CCh, ISO) were performed by one-way analysis of variance (ANOVA). Student's unpaired t-test was used to compare the affects of different agents (PBS, LPS, FIZZ1). A p-value of less than 0.05 was considered significant.

### Example 10

### TSM Contractile Response and Airway Inflammation in OA-Treated Mouse Model

Experiments in this example were directed to characterizing a mouse model for airway hyperresponsiveness (AHR). In preliminary experiments, a mouse model for AHR was established based on the observation that a 10-day OA challenge is able to model abnormal functional behavior of TSM in response to electric field stimulation (Matsubara et al. “Inhibition of Spleen Tyrosine Kinase Prevents Mast Cell Activation and Airway Hyperresponsiveness,” Am J Respir Crit. Care Med. (2006) 173:56-63).

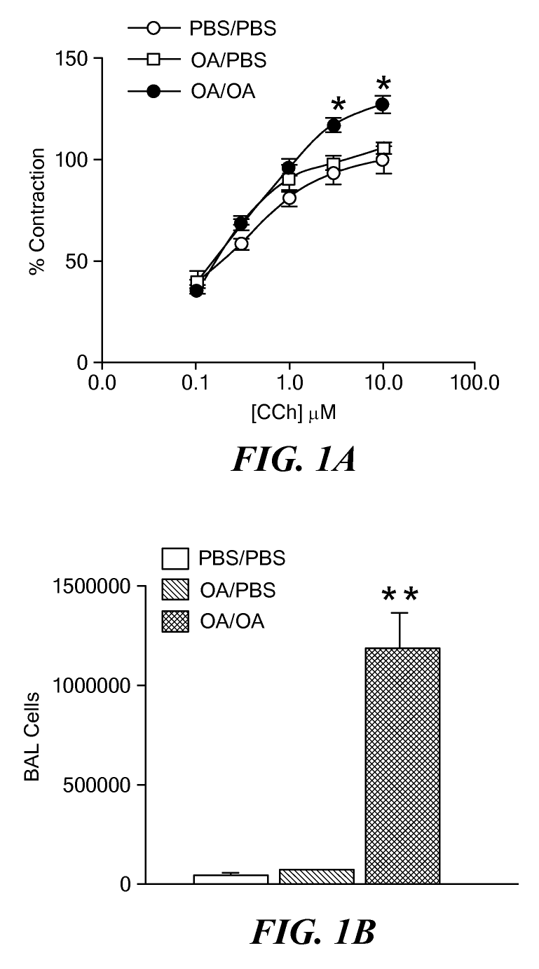

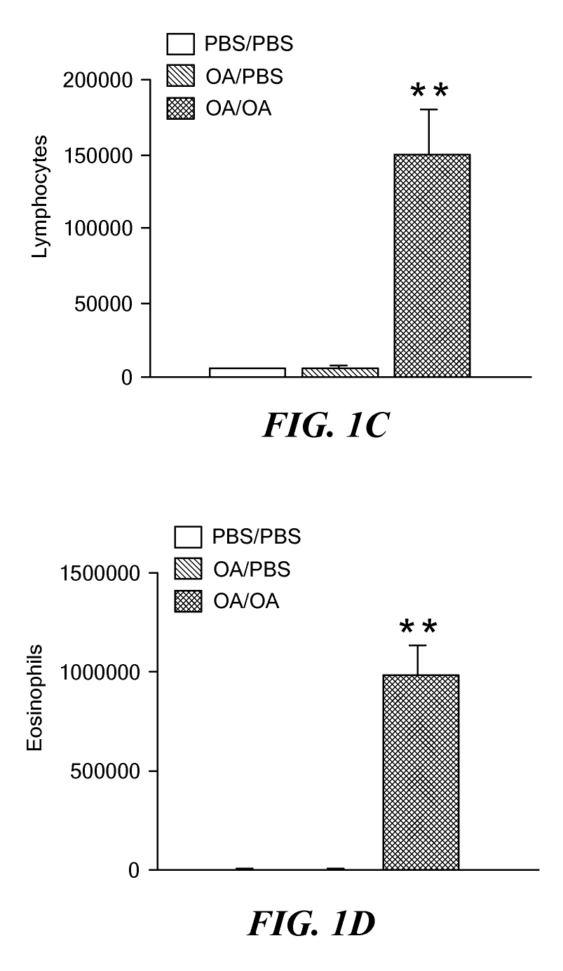

CCh produces a potent contractile response with a concentration-dependent increase in isometric tension of TSM. In vitro responsiveness of TSM to CCh was first examined in trachea from mice receiving a treatment of either PBS/PBS, OA/PBS or OA/OA (FIG. 1A). The contractile response of tracheal rings to CCh was increased in the OA/OA-treated mice as compared with those of the PBS/PBS- and the OA/PBS-treated animals. The contractile forces (%) of TSM were shown as 100±6.77, 105.21±2.71 and 127.75±3.54 in PBS/PBS-, OA/PBS-, and OA/OA-treated mice, respectively. The difference in the level of CCh-evoked force generation was found to be statistically significant (P<0.05, n=6) when comparing either PBS/PBS vs. OA/OA or OA/PBS vs. OA/OA.

Cellular composition of the BAL was determined for PBS/PBS-, OA/PBS-, and OA/OA-treated mice (FIG. 1B-D). A large increase in the number of total BAL cells was clearly observed in the OA/OA-treated mice. The number of lymphocytes and eosinophils in the BAL from OA/OA-treated mice was markedly increased as compared to those from the other two groups. The differences in all the cellular counts between either PBS/PBS vs. OA/OA or OA/PBS vs. OA/OA were statistically significant (P<0.01, n=6).

These results indicate that this animal model is associated with a significant increase in CCh-evoked force and a large inflammatory infiltrate, comprised mainly of lymphocytes and eosinophils, into the BAL, similar to those seen in patients with asthma.

### Example 11

### Levels of FIZZ1 mRNA and Protein in OA-Treated Mouse Model

The experiments in this Example 11 were directed to identifying proteins that may play a role in airway hyperresponsiveness. FIZZ1 was identified in transcriptional profiling experiments.

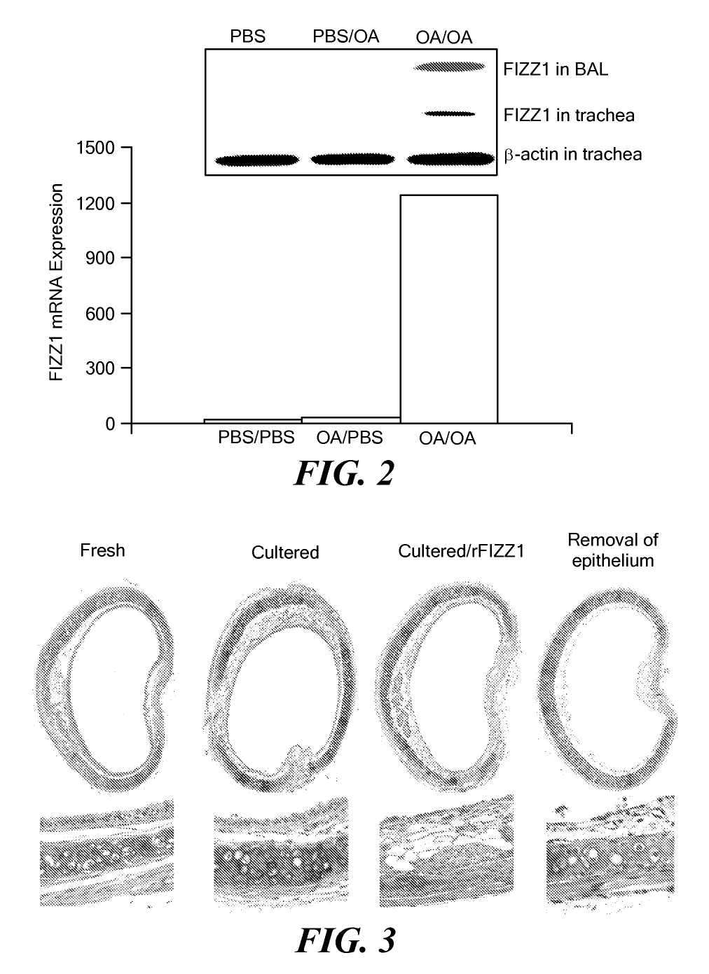

Levels of FIZZ1 mRNA expression in tracheal tissue were examined by transcriptional profiling. Profiling data was filtered and significant differences were determined in the level of mRNA expression using a one-way ANOVA (FIG. 2). The fold change (Fc) in FIZZ1 mRNA expression was calculated for trachea from naïve mice compared to mice treated with either PBS/PBS, PBS/OA or OA/OA. The Fc in FIZZ1 mRNA expression in trachea from mice treated with OA/OA was increased a 1000-fold over that from either PBS/PBS- or OA/PBS-treated mice.

In association with the FIZZ1 mRNA expression data, Western blot analysis of expression of FIZZ1 protein was performed on BAL and trachea from mice treated with either PBS/PBS, OA/PBS or OA/OA (FIG. 2). In contrast to the inability to measure FIZZ1 protein in the BAL from either PBS/PBS- or OA/PBS-treated mice, FIZZ1 protein was easily detected in the BAL from the OA/OA-treated mice.

These results identify FIZZ1 as one of the early phase gene products induced during the initial stage of allergen-triggered airway inflammation. In addition, detection of FIZZ1 protein in BAL and in trachea of OA/OA-treated mice suggests that FIZZ1 may have a role as a proinflammatory mediator propagating allergic inflammation. Without wishing to be bound by any particular theory, the correlation of increased FIZZ1 protein expression and the induction of hyperresponsiveness in inflamed trachea suggests that FIZZ1 contributes to a cascade of effects culminating in TSM dysfunction.

### Example 12

### Histological Analysis of Tracheal Rings and Epithelium

FIZZ1 is one of many pro-inflammatory protein mediators found in airway epithelium (Holcomb et al., “FIZZ1, a novel cysteine-rich secreted protein associated with pulmonary inflammation, defines a new gene family,” The EMBO Journal (2000) 19:4046-4055 and Teng et al., “FIZZ1/RELMα, a novel hypoxia-induced mitogenic factor in lung with vasoconstrictive and angiogenic properties,” Circ Res (2003) 92: 1065-1067), suggesting that FIZZ1 protein exerts its effect on the local environment. In this example, effect(s) of FIZZ1 on its local environment was examined by histological examination of the airway epithelium.

Fresh trachea and trachea cultured in DMEM overnight were examined in whole and sectional tissues in absence and presence of either FIZZ1 protein (100 nM) or LPS (0.1 ng/mL). Examination by light microscope at ×4.0 and ×20.0 magnification showed no tissue edema, unusual epithelial denudation and/or patchy shedding of epithelial cells on the luminal side of the PBS-cultured trachea (FIG. 3). In contrast to trachea cultured with PBS, the epithelial layer in the tracheal rings treated with rFIZZ1, but not with LPS (data not shown), was thinner and some of airway epithelium was denuded, lacking histological intactness. However, the smooth muscle layers in PBS, FIZZ1, and LPS cultured groups were clear and histologically intact. To assess the effect of denuded epithelium on the tracheal response to rFIZZ1, epithelial cells lining the lumen of the tracheal rings were mechanically removed by gently rubbing the intraluminal surface. The status of the epithelium is shown in FIG. 3. Histopathological results of the removal of the luminal epithelium showed a similar state of epithelial denudation to that seen in trachea treated with rFIZZ1. By light microscopy, most epithelium in the trachea was not intact and patchy shedding of the epithelial cells was observed. Some of the epithelial layer was isolated from the basal membrane and released into the luminal side in the cultured trachea.

These histological analyses demonstrated that the epithelial layer was significantly thinner and lacked histological intactness with epithelial denudation in FIZZ1-treated rings. Thinning of the epithelial layer is not caused by serum in the media because histological changes seen in cultured trachea were identical to those seen in fresh trachea.

The results reveal that FIZZ1 acts on airway epithelial tissue and leads to loss of the epithelial barrier. Epithelial damage is clinically associated with human asthmatic disease. Epithelial damage has been accepted as one of the features of the pathogenesis of asthma (Laitinen et al., “Damage of the Airway Epithelium and Bronchial Reactivity in Patients with Asthma,” Am Rev Respir Dis (1985)13:599-606 and Holgate et al., “The epithelium takes centre stage in asthma and atopic dermatitis,” Trends in immunology (2007) 28:248-250). Since epithelial damage in asthma is often caused by a release of major basic proteins from infiltrating inflammatory airways (Motijima et al. “Toxicity of eosinophil cationic proteins for guinea pig tracheal epithelium in vitro,” Am Rev Respir Dis (1989) 139:801-805), data from this Example indicates that FIZZ1 is likely to be one of these basic proteins that cause damage to the airway epithelium. Based on these histological findings, it is likely that changes observed in vitro in the epithelial tissue from the FIZZ1-exposed trachea mirrors that of the in vivo asthmatic airway.

### Example 13

### Effect of rFIZZ11 Heat-Inactivated rFIZZ11 and LPS on CCh-Induced Contraction

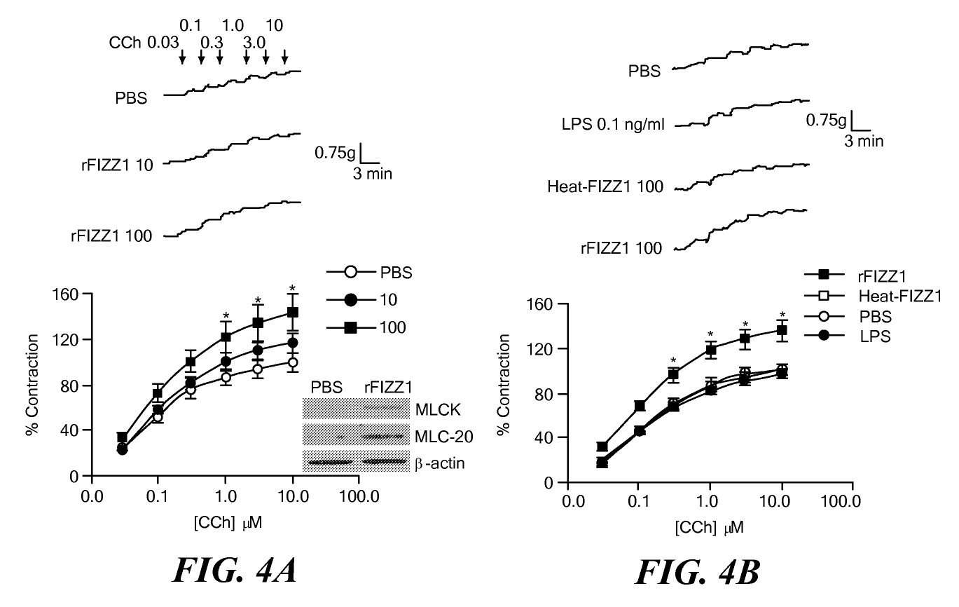

To determine what effect recombinant FIZZ1 protein has on ex vivo contraction of TSM to CCh, mouse tracheal rings treated with FIZZ1 at different concentrations were examined for CCh-evoked force generation. Mouse tracheal rings treated with 100 nM FIZZ1 showed an increased contractile response to CCh as compared to PBS-treated rings (FIG. 4A). Original tracings for CCh-evoked force generation in PBS- and rFIZZ1-treated trachea are shown (FIG. 4A, upper panel). Maximal tensions (%) were 100±8.39 in PBS-treated rings, and 117.21±7.87 and 144.16±15.77 in 10 and 100 nM rFIZZ1-treated trachea, respectively (FIG. 4A, lower panel). The difference in the CCh-induced force generation between PBS- and 100 nM FIZZ1-treated groups was statistically significant (P<0.05, n=6). In support of this finding, expression levels of MLCK and a product of MLCK-phosphorylation, MLC-20, were measured by western blot analysis. An increase in the expression of both MLCK and MLC-20 was found in FIZZ1-treated trachea as compared to PBS-treated trachea (FIG. 4A). Table 5 shows LogEC₅₀ values for TSM sensitivities to the agonist in presence or absence of FIZZ1 (10 and 100 nM). There were no significant differences in LogEC₅₀ values between PBS- and either 10 nM or 100 nM FIZZ1-treated groups as determined by the Student's unpaired T test (P>0.05, n=6).

To verify that the effect of FIZZ1 on CCh-evoked force generation was due to native folded rFIZZ1 protein, tracheal rings were cultured with 100 nM heat-inactivated rFIZZ1 or 0.1 ng/mL LPS and their effects on CCh-evoked TSM contractile response were measured (FIG. 4B). Heat-inactivated FIZZ1 had no observable effect on CCh-evoked force generation as compared to that of native rFIZZ1 protein (FIG. 4B, lower panel). The maximal responses (%) of the tracheal rings were 100.00±4.77 and 135.67±9.02 in the heat inactivated rFIZZ1- and native rFIZZ1-treated groups (P<0.05 by Student's unpaired t-test, n=6), and 100±4.82 and 96.40±4.31 in PBS- and LPS-treated groups (P>0.05 by Student's unpaired t-test, n=6), respectively.

TABLE 5
TSM sensitivity to CCh (Mean ± SE)

| Groups | LogEC₅₀ (μM) | N |
|---|---|---|
| PBS | −0.96 ± 0.07 | 6 |
| FIZZ1 (10 nM) | −0.96 ± 0.05 | 6 |
| FIZZ1 (100 nM) | −0.99 ± 0.03 | 6 |

These results show that rings treated with 100 nM but not 10 nM FIZZ1 had a significant increase in the CCh-generated force without affecting TSM sensitivity to the agonist. In support of this result, expression levels of MLCK and its primary substrate, MLC-20, were examined and found to have significantly increased protein expression in FIZZ1-treated trachea compared to PBS-treated trachea. This finding identifies an important molecular basis underlying the force development observed in the FIZZ1-treated trachea and supports the conclusion that FIZZ1 alters contractile proteins within the tissue.

### Example 14

### Effect of rFIZZ1 on ISO-Induced Relaxation

The overall contractile response of TSM is a summation of both the contractile and the relaxation response of the tissue. In order to address the possibility of an imbalance between these two force responses in the TSM, ISO-induced relaxation was examined in tracheal rings incubated with rFIZZ1. ISO is an agonist of β2-AR and can induce TSM relaxation at a level of 50% of the relaxation by papaverine in either presence or absence of rFIZZ1. Experiments in this Example were conducted to evaluate whether the increase in CCh-evoked TSM force generation after culturing with rFIZZ1 was due to an increased contractile response or a decreased relaxation response in the smooth muscle.

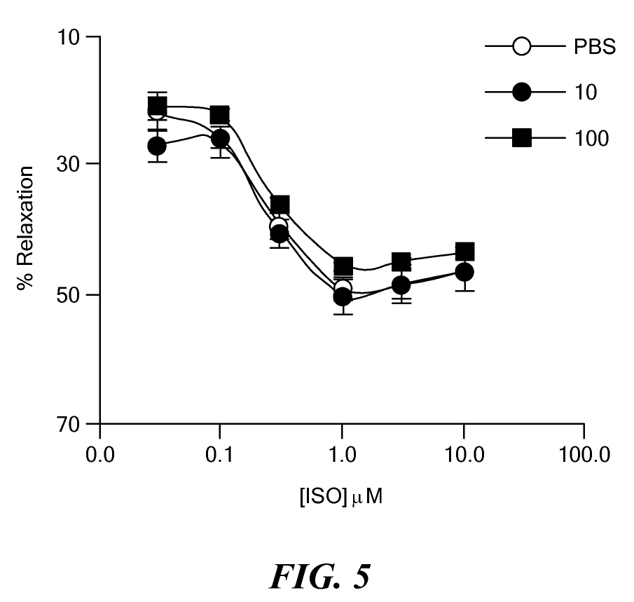

For reference, the degree of TSM relaxation induced by ISO was normalized to the maximal relaxing response induced by 200 μM of papaverine. The effect of pretreatment with either 10 nM or 100 nM rFIZZ1 on the ISO-mediated maximal relaxant forces was measured (FIG. 5). The ISO-induced relaxation of TSM was not affected by pre-incubation with either 10 nM or 100 nM rFIZZ1. The values of the maximal relaxing force (%) for the different groups of treatment were found to be 46.29±2.85, 46.52±2.80 and 43.38±0.75 for the rings treated with PBS, 10 nM and 100 nM rFIZZ1, respectively. None of the differences in these values were statistically significant different as determined by the Student's paired t-test (P>0.05, n=6).

These results demonstrate that rFIZZ1 did not influence the ISO-mediated relaxation response in the rings. It is known that ISO relaxes TSM through a cAMP-dependent protein phosphorylation cascade in a nearly ubiquitous system via an activation of β2-AR-coupled Gs protein, resulting in an increase in adenylate cyclase activity (Knox et al., “Airway smooth muscle relaxation,” Thorax (1995) 50:894-901). Based on the observation that protein expression of FIZZ1 is upregulated in inflamed tissue and that it acts predominantly on the contractile apparatus, FIZZ1 could be a useful therapeutic target for the treatment of AHR in asthma patients.

### Example 15

### Force Response of Fresh Tracheas and Counts of BAL Cells in rFIZZ1-Challenged Mice

Experiments described in this Example were directed toward validating in vivo the observed effect of the mouse FIZZ1 protein on cultured trachea.

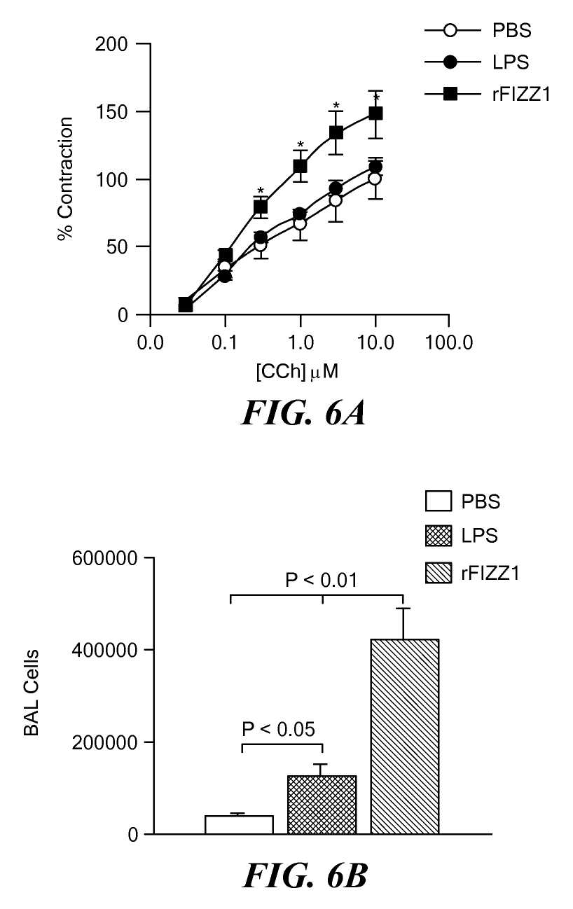

Tensions of fresh tracheal rings and counts of BAL cells were examined 24 hours after the last treatment in mice receiving a series of intranasal doses of PBS, 0.1 ng/ml LPS or 100 nM rFIZZ1 (once a day×5 days). The results are shown in FIG. 6. A significant (P<0.05, N=5) increase in the CCh-evoked force response (A) measured in the fresh trachea and in the number of BAL cells (B) in the lavage was detected in rFIZZ1-challenged mice compared to either PBS- or LPS-challenged mice. Maximal tensions (%) of TSM were 100.00±14.63, 108.28±5.44 and 147.78±18.57 in the PBS-, LPS- and rFIZZ1-exposed groups, respectively. In addition, an obvious increase in the cell counts but not the force response was observed in LPS-treated mice compared to PBS-treated animals (P<0.05).

These results indicate that FIZZ1 protein participates in modulating airway inflammation and TSM activity. A large increase in FIZZ1 protein was observed in vivo in OA-sensitized and challenged mice and an increased force response was measured in fresh trachea from ice treated with in vivo-delivered rFIZZ1 protein. Such observations strongly support the pathophysiological relevance of the phenomenon occurring in cultured trachea and suggests a role for endogenous FIZZ1 protein in regulating airway inflammation and TSM tone in diseased tissues as well.

### Example 16

### Effect of rFIZZ1 on Airway Epithelium

The airway epithelium is a target of physical and allergic insults. Experiments described in this Example were based in part on the finding of epithelial denudation in FIZZ1-treated trachea and were conducted to confirm the effect of rFIZZ1 on airway epithelium.

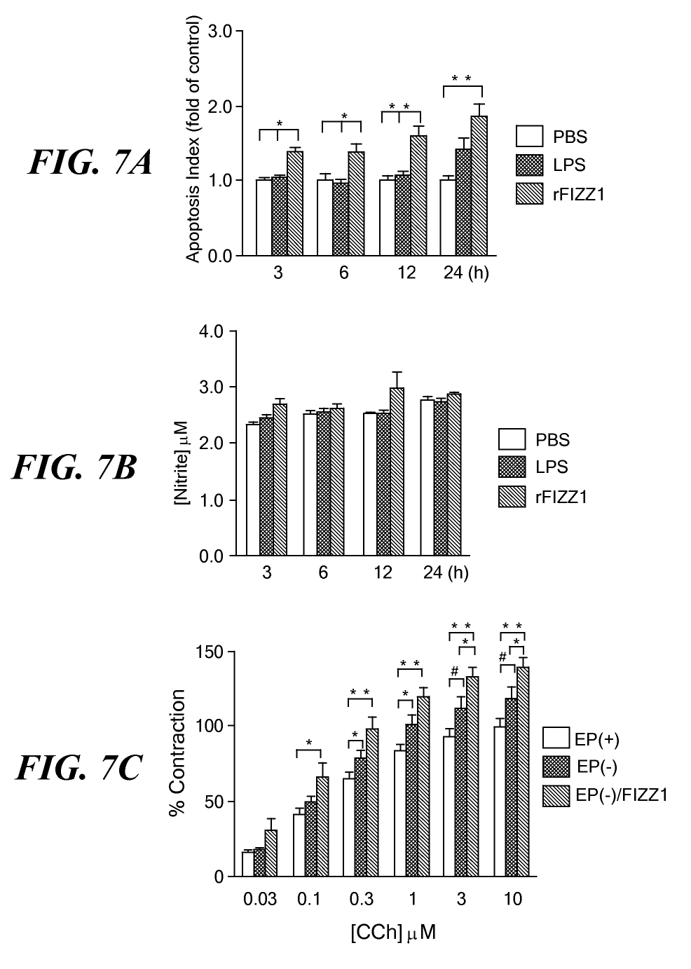

To evaluate possible mechanisms of FIZZ1-mediated loss of the epithelial cell layer, apoptosis and nitrate concentration were measured. The apoptosis index and nitrite concentration in supernatants from treated MTEC were measured using Cell Death Detection ELISA^{plus} and the Griess reaction, respectively. Levels of cytoplasmic histone-associated-DNA-fragments and nitrite concentration in the culture supernatants were measured at the indicated time points. Results are shown in FIG. 7A, B. A significant increased (P<0.05 or 0.01; N=3) in the MTEC apoptosis was detected at all of the time points after rFIZZ1 treatment compared to LPS treatment. The changes in nitrite concentration, however, were not obvious among the three groups at any of the measured time points (N=3).

These results show a significant increase in cell death in FIZZ1-treated cells, initiating at 3 hours of incubation with FIZZ1. Without wishing to be bound by any particular theory, it is suggested that FIZZ1 acts in a complex manner on airway tissues with its initial inflammatory effect contributing to epithelial dysfunction. It is known that NO is synthesized in airway epithelium and acts on TSM cells (Barnes and Belvisi, “Nitric oxide and lung disease,” Thorax (1993) 48:1034-1043). Since there were no obvious changes in nitrite levels from any of the experimental groups, without wishing to be bound by any particular theory, it is contemplated that NO is not involved in the observed changes in the TSM force response nor in the loss of the epithelial layer.

In order to clarify whether epithelial damage contributes to an increased force response in FIZZ1-exposed trachea, TSM tension was examined in trachea with epithelial denudation compared to that with intact epithelium. Contractile responses and sensitivities of TSM to CCh stimulation are shown in FIG. 7C and Table 6. The maximal tensions (%) were 100±6.22, 119.30±8.16 and 141.43±6.65 in trachea with intact epithelium (EP(+)) and trachea with denuded epithelium (EP(−)) treated with and without 100 nM FIZZ1, respectively. There were significant differences (P<0.05, n=8, 19) in the maximal contractile response of TSM from EP(−) and EP(−)/FIZZ1 trachea (P<0.05) and there were significant difference in force generation at the doses of CCh used between EP(+) and EP(−) as determined by the Student's paired t-test (P<0.05). The LogEC₅₀ values of TSM were calculated and showed no obvious change in sensitivities between any two groups.

TABLE 6
EP(−) TSM sensitivity to CCh (Mean ± SE)

| Groups | LogEC₅₀ (μM) | N |
|---|---|---|
| EP(+) | −0.85 ± 0.04 | 19 |
| EP(−) | −087 ± 0.03 | 19 |
| EP(−)/FIZZ1 100 nM | −0.91 ± 0.08 | 8 |

These results show an increased force response in epithelium-denuded trachea, indicating a possible importance of the epithelial barrier in protecting TSM from direct exposure to an agonist in a contractile response. Epithelium-denuded trachea treated with rFIZZ1 showed a marked increase in the force level compared to that of denuded trachea with no treatment, indicating that this protein mediator exerts separable effects involving both the epithelium and TSM tissues. Without wishing to be bound by any particular theory, it is contemplated that the dual effects of FIZZ1 represent different stages in the process of abnormal smooth muscle force development.

### Example 17

### Protein Expression in FIZZ1-Treated Trachea

Many signal transduction molecules, including pro-inflammatory proteins, are involved in the transformation of a receptor/ligand binding event into TSM contraction. The experiments in this Example were conducted to determine the effect rFIZZ1 would have on certain signaling intermediates involved in TSM contraction.

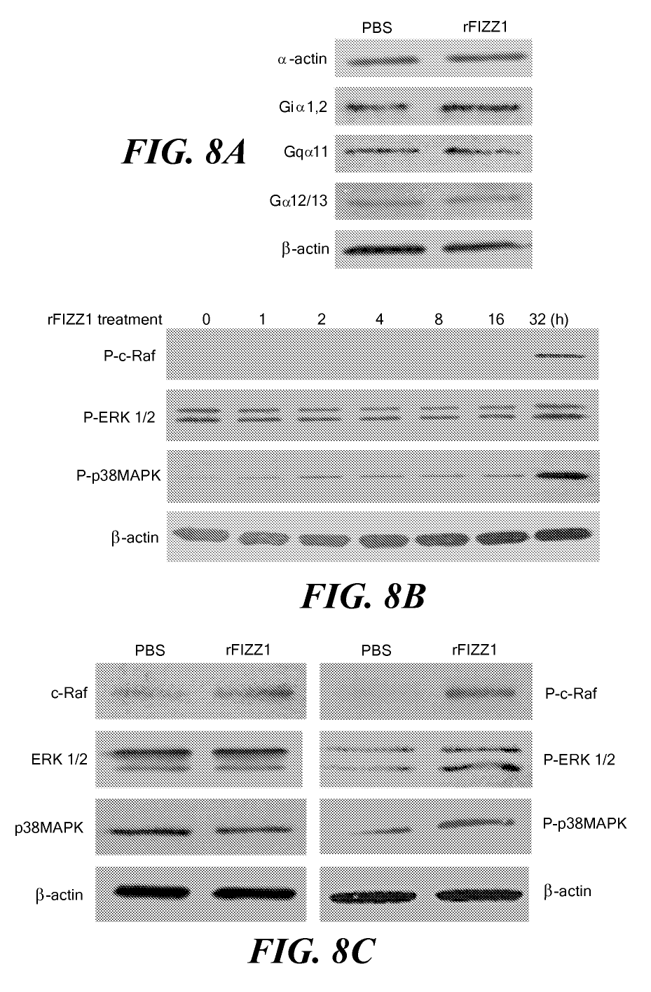

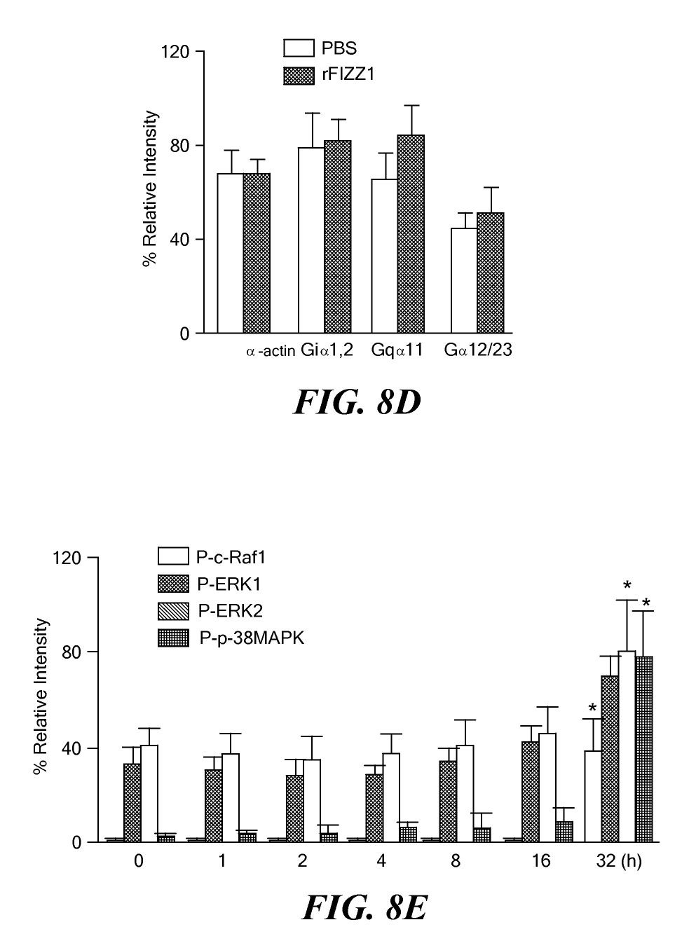

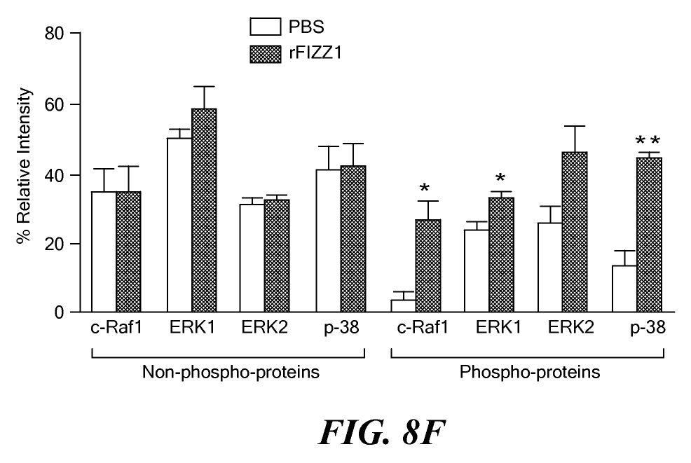

Protein expression levels of α-actin, G proteins such as Giα1,2, Gqα11, Gα12/13 and several proteins involved in the MAPK pathway (i.e., c-Raf, phospho-c-Raf, ERK1/2, phospho-ERK1/2, p38 MAPK and phospho-p38 MAPK) were examined in tissue lysates from either PBS-treated or rFIZZ1-treated trachea using western blot analysis (FIG. 8). The protein expression levels for α-actin and all the G proteins tested were similar between the PBS-treated and the rFIZZ1-treated trachea when normalized to the level of expression of β-actin in same tissue (FIG. 8A). In contrast to these protein expression levels, the phosphorylation state of proteins involved in the MAPK pathway, such as c-Raf, ERK1/2 and p38 MAPK, showed a marked time-dependent increase in their level of phosphorylation upon treatment with rFIZZ1 (FIG. 8B). This phosphorylation displayed a slow kinetic profile, greatly increasing only between 16 and 32 hours after the addition of FIZZ1. A comparison of the expression at 24 hours between PBS-treated and FIZZ1-treated trachea of phosphorylated and unphosphorylated c-Raf, ERK1/2 and p38 MAPK showed a similar level of expression for most of the unphosphorylated proteins in both treatment groups (FIG. 8C). The exception was c-Raf, whose expression was slightly increased after culturing with FIZZ1 compared to the PBS-cultured trachea. In contrast, phospho-c-Raf, phospho-ERK1/2, and phospho-p38 MAPK all increased in expression 24 hours after rFIZZ1 treatment compared to PBS treatment. The relative intensity (%) of the quantified band for each of the phosphorylated proteins at 24 hours was calculated in reference to the intensity of β-actin (FIG. 8D-F). Compared to protein expression in PBS-treated trachea, there were statistically significant (P<0.01 or 0.05 vs PBS, n=3) increases in the expression levels of phospho-c-Raf, phospho-ERK1/2 and phospho-p38 MAPK in rFIZZ1-treated tissues. Likewise, a similar significant increase in the phosphorylation of these proteins was observed in the kinetic profile at the 32-hour post rFIZZ1 time point (FIG. 8E).

These results show that α-actin is expressed at a similar level in both rFIZZ1- and PBS-treated tissues, indicating that FIZZ1 is unlikely to exert its effects by directly changing the expression of this contractile element in this system.

Furthermore, these results show that FIZZ1 treatment induces not only high levels of phospho-ERK1/2 and phospho-p38 MAPK but also high levels of phospho-c-Raf expression in tracheal rings, suggesting that FIZZ1 is sufficient to cause an activation of this arm of the MAPK signaling pathway in ex vivo tracheal organ cultures. Without wishing to be bound by any particular theory, it is contemplated that FIZZ1 regulation of the CCh-evoked force is likely to act through the c-Raf-linked MAPK signaling cascade, leading to an increase in MLC-20 phosphorylation in contracted TSM.

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all, of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects are excluded are not set forth explicitly herein.

### INCORPORATION OF REFERENCES

All publications and patent documents cited in this application are incorporated by reference in their entirety to the same extent as if the contents of each individual publication or patent document were incorporated herein.

What is claimed is:

1. A method to reduce airway hyperresponsiveness in a mammal, the method comprising a step of decreasing Found in Inflammatory Zone (FIZZ1) activity.

2. The method of claim 1, wherein the step of decreasing the FIZZ1 activity comprises reducing transcription of FIZZ1 gene.

3. The method of claim 1, wherein the step of decreasing the FIZZ1 activity comprises reducing translation of an mRNA sequence encoding FIZZ1 protein.

4. The method of claim 1, wherein the step of decreasing the FIZZ1 activity comprises administering an interfering RNA.

5. The method of claim 4, wherein the interfering RNA is selected from siRNA, shRNA or miRNA.

6. The method of claim 5, wherein the interfering RNA is siRNA.

7. The method of claim 6, wherein said siRNA comprises a sequence substantially complementary to at least a portion of the mRNA encoding the FIZZ1 protein.

8. The method of claim 6, wherein said siRNA is double-stranded.

9. The method of claim 6, wherein said siRNA is single-stranded.

10. The method of claim 6, wherein said siRNA comprises a sequence having between about 20 and about 25 nucleotide bases.

11. The method of claim 1, wherein the step of decreasing the FIZZ1 activity comprises administering an antibody, or a fragment thereof, that specifically binds the FIZZ1 protein.

12. The method of claim 10, wherein the antibody, or a fragment thereof, is selected from the group consisting of intact IgG, F(ab′)2, F(ab)₂, Fab′, Fab, ScFvs, diabodies, triabodies and tetrabodies.

13. The method of claim 12, wherein the antibody is a monoclonal antibody.

14. The method of claim 13, wherein the antibody is a humanized monoclonal antibody.

15. The method of claim 1, wherein the step of decreasing the FIZZ1 activity comprises administering an aptamer that specifically binds the FIZZ1 protein.

16. The method of claim 15, wherein the aptamer is an RNA aptamer.

17. The method of claim 1, wherein the step of decreasing the FIZZ1 activity comprises administering a small molecule that inhibits the FIZZ1 activity.

18. The method of claim 1, wherein the step of decreasing the FIZZ1 activity comprises reducing the FIZZ1 activity in tracheal smooth muscle of the mammal.

19. The method of claim 1, wherein the step of decreasing the FIZZ1 activity comprises reducing the FIZZ1 activity in airway epithelium.

20. The method of claim 1, wherein the airway hyperresponsiveness is associated with asthma.

21. A method for treating inflammation, the method comprising a step of decreasing FIZZ1 activity in a mammal in need of the treatment.

22-44. (canceled)

45. A method for evaluating the ability of an agent to modulate airway inflammation, the method comprising the steps of:
(1) providing a trachea sample;
(2) culturing the trachea sample in a medium in the presence of FIZZ1;
(3) providing an agent to the medium;
(4) determining the histology of the trachea sample; and
(5) comparing the histology result from step (4) to a control to evaluate the ability of the agent to modulate airway inflammation.

46-50. (canceled)

51. A method for evaluating the ability of an agent to modulate airway hyperresponsiveness, the method comprising the steps of:
(1) providing a trachea sample;
(2) culturing the trachea sample in a medium in the presence of FIZZ1;
(3) providing an agent to the medium;
(4) providing carbachol to the medium;
(5) determining a contractile response to carbachol of the trachea sample; and
(6) comparing the contractile response to carbachol determined in step (5) to a control to evaluate the ability of the agent to modulate airway hyperresponsiveness.

52-54. (canceled)

55. A method of screening inhibitors of FIZZ1, the method comprising the steps of:
(1) providing a plurality of trachea samples, each of which is cultured in a medium in the presence of FIZZ1;
(2) providing a plurality of inhibitor candidates;
(3) determining a phenotype associated with FIZZ1-mediated airway inflammation or hyperresponsiveness in each of the plurality of trachea samples;
(4) comparing the phenotype determined in step (3) to a control; and
(5) identifying one or more inhibitors of FIZZ1 that reduce the phenotype based on the comparison result in step (4).

56-63. (canceled)

64. An inhibitor of FIZZ1 identified by the method of claim 56.

65. A small molecule inhibitor of FIZZ1 identified by the method of claim 57.

66. A method for enhancing an immune response in a mammal, the method comprising administering a polypeptide encoding FIZZ1 protein (SEQ ID NO:4), a fragment thereof, or a variant having at least 90% sequence identity to the FIZZ1 protein (SEQ ID NO:4).

67. A vaccine comprising a polypeptide encoding FIZZ1 protein (SEQ ID NO:4), a fragment thereof, or a variant having at least 90% sequence identity to the FIZZ1 protein (SEQ ID NO:4).
